(19) United States
(12) Patent Application Publication
(10) Pub. No.: US 2015/0099947 A1
(43) Pub. Date: Apr. 9, 2015

(54) SKIN YOUTHFULNESS INDEX, METHODS AND APPLICATIONS THEREOF

(71) Applicant: ACCESS BUSINESS GROUP INTERNATIONAL LLC, Ada, MI (US)

(72) Inventors: Di Qu, Ada, MI (US); Yulia Park, Grand Rapids, MI (US)

(73) Assignee: ACCESS BUSINESS GROUP INTERNATIONAL LLC, Ada, MI (US)

(21) Appl. No.: 14/495,497

(22) Filed: Sep. 24, 2014

Related U.S. Application Data
(60) Provisional application No. 61/887,024, filed on Oct. 4, 2013.
(60) Provisional application No. 61/991,092, filed on May 9, 2014.

(51) Int. Cl. A61B 5/00

(52) U.S. Cl. CPC A61B 5/442; A61B 5/0064; A61B 5/0077; A61B 5/444; A61B 5/4848; A61B 5/6898; A61B 5/7278; A61B 5/742; A61B 5/7275; A61B 5/443; A61B 2576/02
USPC 600/306

(57) ABSTRACT

The present invention relates to a Skin Youthfulness Index and methods of determining the same. The present invention also relates to methods of determining an apparent age of a subject and to methods for measuring an improvement of facial skin characteristics following a treatment by comparing the Skin Youthfulness Index values before and after the treatment.

### RELATED APPLICATIONS

The present patent document claims the benefit of the filing date under 35 U.S.C. §119(e) of Provisional U.S. Patent Application Ser. No. 61/887,024, filed Oct. 4, 2013 and of Provisional U.S. Patent Application Ser. No. 61/991,092, filed May 9, 2014, the entire contents of which are hereby incorporated by reference.

### BACKGROUND

The present invention relates to a Skin Youthfulness Index (SYI) and its uses and methods.

Skin is the largest organ in the human body and measuring the changes in its properties with age is a primary topic in skin related research [Yaar, M., Clinical and Histological Features of Intrinsic Versus Extrinsic skin Aging, in: Gilchrest, B. A. and Krutmann, J. (Eds.), Skin Aging, Springer, Berlin, Heidelberg, 2006, pp. 9-21].

Beauty-conscious consumers desire to maintain a youthful skin appearance of skin. Therefore, it is important to those consumers to know the condition of their skin to be able to choose a skin care regimen or skin treatment appropriate to their daily needs and/or desired results.

Although humans are generally able to characterize a person to be within a particular age group based on an image of the person's age, more accurate ways of determining person's apparent age are highly desirable. As such, skin care researchers have long strived to develop a comprehensive model to correlate visual properties of skin with age that would provide an objective, quantitative description of skin conditions and help assess treatment efficacy of skin products or procedures.

Currently, most models reported in the literature use subjectively measured skin parameters to assess skin aging.

Skin care researchers have strived to develop a comprehensive model correlating multiple skin properties with age thereby providing an objective and quantitative measure of skin conditions that will help assess the efficacy of skin care products and treatments [Lange, N., and Weinstock, M., Statistical Analysis of Sensitivity, Specificity, and Predictive Value of a Diagnostic Test, in: Serup, J., Jemec, G., and Grove, G. L. (Eds.), Handbook of Non-Invasive Methods and the Skin, CRC Press, 2006, 2^{nd} Edition, pp. 53-62].

Guinot et al. introduced a skin age score (SAS) correlating 24 visual and tactile parameters of facial skin with chronological age, concluding that SAS could be generated from the evaluation of multiple discrete signs on facial skin and was an informative tool for quantifying skin aging [Guinot, C., Malvy, D. J., Ambroisine, L., Latreille, J., Mauger, E., Tenenhaus, M., Morizot, F., Lopez, S., Le Fur, I., and Tschachler, E., Relative Contribution of Intrinsic vs Extrinsic Factors to Skin Aging as Determined by a Validated Skin Age Score, Arch. Dermatol. 138 (2002) 1454-1460].

Vierkotter et al. reported a skin aging index (SCINEXA) which incorporates 23 clinically graded intrinsic and extrinsic parameters characteristic of skin aging [Vierkotter, A., Rank U., Kramer, U., Sugiri, D., Reimann, V., and Krutmann, J., The SCINEXA: A Novel, Validated Score to Simultaneously Assess and Differentiate Between Intrinsic and Extrinsic Skin Ageing, J. Dermatol. Sci., 53 (2009) 207-211]. They concluded that the model could be used to separate the extrinsic and intrinsic effects of aging.

Nkengne et al. established an index of aging using clinically graded parameters such as the degree of wrinkles, brown spots, and sagging [Nkengne, A., Roure, R., Rossi, A. B., and Bertin, C., The Skin Aging Index: a New Approach for Documenting Anti-aging Products or Procedures, Skin Res. Technol., 19 (2013) 291-298]. They believed that their skin aging index captured information relevant to the visual transformation of facial skin with age and was meaningful when applied to product efficacy evaluations.

Additional research by Bazin and Doublet described linear correlations with multiple clinically assessed parameters for Caucasian and Asian populations, respectively [Bazin, R. and Doublet, E., Skin Aging Atlas, Volume 1 Caucasian Type, Editions Med'Com, Paris, 2007, pp. 32], and Bazin and Flament [Bazin, R., and Flament, F., Skin Aging Atlas Volume 2 Asian Type, Editions Med'Com, Paris, 2010, pp. 26]. While subjective grading is the current standard of clinical assessment, it is a common belief that the subjectivity of these assessments carries the intrinsic possibility of variation between graders and inconsistency in the grader's perception at different time points.

Zedayko et al. developed an instrumental method to correlate age with skin brightness of Caucasian subjects [Zedayko, T., Azriel, M., and Kollias, N., Caucasian Facial L* Shifts May Communicate Anti-Ageing Efficacy, Int. J. Cosmet. Sci., 33 (2011) 450-454]. While the measurement was objective and the correlation was good, the approach was rather simplistic in that it only used skin color as the measurable aspect of skin aging.

A more complex measurement was established by Dicanio et al., in which a linear function between age and multiple skin parameters were constructed using principal component analysis and multivariate regression [D. Dicanio, D., R. Sparacio, R., L. Declercq, L., H. Corstjens, H., N. Muizzuddin, N., J. Hidalgo, J., P. Giacomoni, P., L. Jorgensen, L., and D. Maes, D., Calculation of apparent age by liner combination of facial skin parameters: a predictive tool to evaluate the efficacy of cosmetic treatments and to assess the predisposition to accelerated aging, Biogerontology, 10 (2009) 757-772]. A total of 76 parameters (10 clinical, 14 biophysical, and 52 biochemical) were analyzed to identify 12 primary variables for age estimation. While the statistical analysis method was sound, the physical significance of their results was still open to discussion. For example, both clinical and instrumental measurements of the same skin property (such as crow's feet) were included in the formula as two independent variables, which was difficult to justify. In addition, both glycation (a biochemical parameter) and the degree of wrinkles (a clinical parameter) were included in their model. Since it is commonly believed that glycation is the molecular marker for the clinical signs of aging [Kollias, N., Gillies, R., Moran, M., Kochevar, I. E., and Anderson, R. R., Endogenous Skin Fluorescence Includes Bands that May Serve as Quantitative Markers of Aging and Photoaging, J. Invest. Dermatol., III (1998) 776-780; and Maillard-Lefebvre, H., Boulanger, E., Daroux, M., Gaxatte, C., Hudson, B. I., and Lambert, M., Soluble Receptor for Advanced Glycation End Products: a New Biomarker in Diagnosis and Prognosis of Chronic Inflammatory Diseases, Rheumatology, 48 (2009) 1190-1196], listing both of them as separate independent variables in a linear equation could potentially impair the validity of the model.

Over the past decade, sophisticated facial imaging systems have been developed to measure visual properties of skin using image analysis [Hawkins, S., Computerized Image Analysis of Clinical Photos, in: Serup, J., Jemec, G., and Grove, G. L. (Eds.), Handbook of Non-Invasive Methods and the Skin, CRC Press, 2006, 2^{nd} Edition, pp. 95-100].

For example, the VISIA™ Complexion System (Canfield Imaging Systems) uses imaging and analysis to capture visual properties of a subject, which are then compared to skin properties of other people of the same age and ethnicity as the subject. The system, however, can only analyze one skin parameter at a time.

In addition, several methods of correlating the conditions of skin with age using multi-variable regression methods are known and used (e.g., U.S. Pat. No. 6,501,982; U.S. Pub. No. 2005/0197542). However, these methods have not been successful because of individual variability between people and incorporating subjectively measured properties of the skin into age correlating methods. The resulting skin age predictions are often unreliable or inaccurate.

As such, improved methods of evaluating individual's skin and correlating the skin parameters to age are highly desirable.

### SUMMARY

Compared to the studies referenced above, which used subjective or multiple instrumental methods to collect age related data, as described herein, exclusive use of image analysis for the quantification of the visual signs of aging has the advantage of being simpler than the multi-instrument method, as well as being more comprehensive than the single measurement technique.

A novel model correlating chronological age of female Asian consumers to a list of objectively measured visual parameters of facial skin is described. This approach establishes a comprehensive function, the Skin Youthfulness Index (SYI), calculated using image analysis to bridge age and the measured skin properties. Special focus was placed on calculating meaningful weight factors for each of the skin parameters in order to improve age correlation and to more accurately predict skin age based on visually displayed skin conditions.

One embodiment relates to a method of determining a Skin Youthfulness Index (SYI) of a subject, comprising measuring visual skin parameters and calculating the SYI of the subject wherein the SYI has an inverse correlation to human age and describes a skin youthfulness of a human subject on a 0 to 100 scale with a higher value corresponding to a more youthful appearance than the chronological age of the subject.

Another embodiment relates to a method of determining a Skin Youthfulness Index (SYI) of a subject, comprising measuring visual skin parameters and calculating the SYI of the subject, wherein the SYI comprises:

SYI=10(10+(0.081 ln T−0.627 ln Wr−0.174 ln P−0.056 ln b*−0.062 ln U) Equation 11

wherein:

T corresponds to translucency index,

Wr corresponds to wrinkle score,

P corresponds to pore score,

b* corresponds to yellowness, and

U corresponds to color unevenness.

Another embodiment relates to a method of determining an SYI of a subject, comprising: measuring visual skin parameters: wrinkle score (Wr), pore score (P), translucency index (T), skin yellowness (b*), and unevenness of skin tone (U) of the subject; normalizing data from the measured visual skin parameters; and calculating the SYI of the subject.

Yet a further embodiment relates to a method for measuring an improvement of skin characteristics following a treatment comprising:
(i) measuring visual skin parameters: wrinkle score (Wr), pore score (P), translucency index (T), skin yellowness (b*), and unevenness of skin tone (U) before (_{b}) and after (ₐ) the treatment;
(ii) determining a before-the treatment skin youthfulness index (SYI_{b}) value, wherein SYI_{b} is calculated:

SYI_{b}=10(10+(0.081 ln T_{b}−0.0627 ln Wrb_{b}−0.174 ln P_{b}−0.056 ln b*_{b}−0.062 ln U_{b}) (Equation 13)
(iii) determining an after-the treatment skin youthfulness index (SYIₐ) value, wherein the SYIₐ is calculated:

SYI_{c}=10(10+(0.081 ln Tₐ−0.0627 ln Wrbₐ−0.174 ln Pₐ−0.056 ln b*ₐ−0.062 ln Uₐ) (Equation 14)
(iv) correlating the SYI_{b} value to an age standard reference curve to determine a before-the treatment age value;
(v) correlating the SYIₐ value to the age standard reference curve to determine an after-the treatment age value; and
(vi) determining the difference between the before-the treatment age value and the after-the treatment age value to measure improvement, if any, of facial skin characteristics following the treatment.

In another embodiment, the present invention relates to a Skin Youthfulness

Index (SYI) that includes:

$$SYI = N {\sum }_{i = 1}^{9} {W}_{i} {V}_{i}^{j} ; \qquad ( Equation 1 )$$

wherein:
W corresponds to a Weight Factor (W);
V corresponds to a value of an objectively measured visual skin parameter (normalized);
i corresponds to a specific type of the measured visual skin parameter:
1=wrinkles, 2=deep layer spots, 3=pores, 4=translucency, 5=skin redness, 6=skin yellowness, 7=Individual Typology Angle (ITA°), 8=unevenness of skin tone, 9=surface textural parameter (entropy);
j=1 for a positive correlation;
j=−1 for a negative correlation; and
N is a factor to target SYI in a 0-100 value.

The Weight Factor (W) may be determined by normalizing a value of an Impact Factor (IF) to a 0-1 scale.

The IF may be determined by:

IF=SF×MIF (Equation 2);

wherein:

SF corresponds to a Significance Factor (SF), wherein a SF value is determined for each individual measured visual skin parameter having a Pearson correlation coefficient greater than a critical value, and where the SF value for each individual measured visual skin parameter is determined by:

SF=r²×r² (Equation 3);

where r² is the coefficient of determination, and

MIF corresponds to Maximum Impact Factor (MIF) and is determined for each individual measured visual skin parameter by:

MIF=(Maximum−Minimum)/Average (Equation 4);

wherein:

Maximum corresponds to a maximum value measured for each individual visual skin parameter;

Minimum corresponds to a minimum value measured for each individual visual skin parameter; and

Average corresponds to an average value for each individual measured visual skin parameter.

A further embodiment relates to a method of determining an SYI of a subject. The method includes measuring one to nine visual skin parameters of the subject; normalizing data from the measured visual skin parameters; calculating the weight factor (W) for the measured visual skin parameters; and calculating the SYI of the subject. The measuring step may be conducted from an image.

The method may further include a step of photographing the subject under five different lighting conditions: standard, flat, UV or narrow-band blue light, cross polarized, and parallel polarized. In the method, the visual skin parameters include wrinkles, deep layer spots, pores, translucency, skin redness, skin yellowness, Individual Typology Angle (ITA°), unevenness of skin tone, and surface textural parameter (entropy). In the methods, the measuring step preferably includes measuring nine visual skin parameters, such as wrinkles, deep layer spots, pores, translucency, skin redness, skin yellowness, Individual Typology Angle (ITA°), unevenness of skin tone, and surface textural parameter (entropy) of the subject.

In the method, the SYI may be determined according to:

$$SYI = N {\sum }_{i = 1}^{9} {W}_{i} {V}_{i}^{j} \qquad ( Equation 1 )$$

wherein:

W corresponds to a Weight Factor (W);

V corresponds to a value of objectively measured visual skin parameter (normalized);

i corresponds to a specific type of measured visual skin parameter:

1=wrinkles, 2=deep layer spots, 3=pores, 4=translucency, 5=skin redness, 6=skin yellowness, 7=Individual Typology Angle (ITA°), 8=unevenness of skin tone, 9=surface textural parameter (entropy);

j=1 for a positive correlation;

j=−1 for a negative correlation; and

N is a factor to target SYI in a 0-100 value.

In the method, the Weight Factor (W) may be determined by normalizing an Impact Factor (IF) value in a 0-1 scale.

In the method, the IF may be determined by:

IF=SF×MIF (Equation 2)

wherein:

SF corresponds to a Significance Factor (SF), wherein a SF value is determined for each individual measured visual skin parameter having a Pearson correlation coefficient greater than a critical value, and where the SF value for each individual measured visual skin parameter is determined by:

SF=r²×r² (Equation 3);

where r2 is the coefficient of determination, and

MIF corresponds to Maximum Impact Factor (MIF) and is determined for each of the measured visual skin parameters by:

MIF=(Maximum−Minimum)/Average (Equation 4);

wherein:

Maximum corresponds to a maximum value for each individual measured visual skin parameter;

Minimum corresponds to a minimum value for each individual measured visual skin parameters; and

Average corresponds to an average value for each individual measured visual skin parameter.

Another embodiment relates to a method of determining an apparent age of a subject. The method includes measuring at least one and up to nine visual skin parameters of the subject; normalizing data from each of the measured visual skin parameters; calculating a Weight Factor (W) for the measured visual skin parameters; calculating a Skin Youthfulness Index (SYI) of the subject; correlating the SYI of the subject to a standard reference curve to determine the apparent age of the subject. In the method, the measuring step may be conducted from an image of the subject.

The method may further include a step of photographing the subject under five different lighting conditions: standard, flat, UV or narrow-band blue light, cross polarized, and parallel polarized. In the method, the visual skin parameters include wrinkles, deep layer spots, pores, translucency, skin redness, skin yellowness, Individual Typology Angle (ITA°), unevenness of skin tone, and surface textural parameter (entropy). The measuring step may include measuring one to nine visual skin parameters selected from: wrinkles, deep layer spots, pores, translucency, skin redness, skin yellowness, Individual Typology Angle (ITA°), unevenness of skin tone, and surface textural parameter (entropy) of the subject. In the method, the SYI comprises:

$$SYI = N {\sum }_{i = 1}^{9} {W}_{i} {V}_{i}^{j} \qquad ( Equation 1 )$$

wherein:

W corresponds to a Weight Factor (W);

V corresponds to a value of objectively measured visual skin parameter (normalized);

i corresponds to a specific type of measured visual skin parameter:

1=wrinkles, 2=deep layer spots, 3=pores, 4=translucency, 5=redness, 6=yellowness, 7=Individual Typology Angle (ITA°), 8=unevenness of skin tone, 9=surface textural parameter (entropy);

j=1 for a positive correlation;

j=−1 for a negative correlation; and

N is a factor to target SYI in a 0-100 value.

The weight factor (W) may be determined by normalizing an Impact Factor (IF) value in a 0-1 scale, wherein the IF is determined by:

IF=SF×MIF (Equation 2);

wherein:

SF corresponds to a Significance Factor (SF), wherein a SF value is determined for each individual measured visual skin parameter having a Pearson correlation coefficient greater than a critical value, and where the SF value for each individual measured visual skin parameter is determined by:

SF=r²×r² (Equation 3);

where r² is the coefficient of determination, and

MIF corresponds to Maximum Impact Factor (MIF) and is determined for each of the measured visual skin parameters by:

MIF=(Maximum−Minimum)/Average (Equation 4);

wherein:

Maximum corresponds to a maximum value for each individual measured visual skin parameter;

Minimum corresponds to a minimum value for each individual measured visual skin parameter; and

Average corresponds to an average value for each individual measured visual skin parameter.

Yet another embodiment relates to a method for measuring an improvement or evaluating effectiveness of skin characteristics following a treatment. The method includes measuring visual skin parameters: wrinkles, deep layer spots, pores, translucency, skin redness, skin yellowness, Individual Typology Angle (ITA°), unevenness of skin tone, and/or surface textural parameter (entropy) before and after the treatment; determining a before-the treatment skin youthfulness index (SYI_{b}) value; determining an after-the treatment skin youthfulness index (SYIₐ) value; correlating the SYI_{b} value to an age standard reference curve to determine a before-the treatment age value; correlating the SYIₐ value to the age standard reference curve to determine an after-the treatment age value; and determining the difference between the before-the treatment age value and the after-the treatment age value to measure improvement, if any, of facial skin characteristics following the treatment. In the method, the measuring step may be conducted from an image.

The method may further include photographing the subject under five different lighting conditions: standard, flat, UV or narrow-band blue light, cross polarized, and parallel polarized. The treatment may be a cosmetic treatment. The treatment may be a medical treatment.

Certain other embodiments relate to a method of determining an apparent age of a subject using a smart device. The method includes using a camera on a smart device to acquire an image of a subject; sending the acquired image to a processor; using a set of instructions that, upon execution by the processor, causes the processor to perform at least the following: (i) using the acquired image to measure visual skin parameters, (ii) determining a before-the treatment skin youthfulness index (SYI_{b}) value, (iii) determining an after-the treatment skin youthfulness index (SYIₐ) value, (iv) correlating the SYI_{b} value to an age standard reference curve to determine a before-the treatment age value, (v) correlating the SYIₐ value to the age standard reference curve to determine an after-the treatment age value, and (vi) determining the difference between the before-the treatment age value and the after-the treatment age value to measure improvement, if any, of facial skin characteristics following the treatment. The method also includes a step of using a display to display a result of the determined difference.

Another embodiment relates to a smart device that includes a display and a computer-readable medium having a set of instructions that, upon execution by a processor, causes the processor to perform at least the following: (i) using one or more images of a subject to measure visual skin parameters, (ii) determining a before-the treatment skin youthfulness index (SYI_{b}) value, (iii) determining an after-the treatment skin youthfulness index (SYIₐ) value, (iv) correlating the SYI_{b} value to an age standard reference curve to determine a before-the treatment age value, (v) correlating the SYIₐ value to the age standard reference curve to determine an after-the treatment age value, (vi) determining the difference between the before-the treatment age value and the after-the treatment age value to measure improvement, if any, of facial skin characteristics following the treatment. The method also includes a step of causing the display to display a result of the determined difference.

### BRIEF DESCRIPTION OF THE DRAWINGS

The patent or application file contains at least one drawing (color photographs) executed in color. Copies of this patent or patent application publication with color drawing(s) will be provided by the Office upon request and payment of the necessary fee.

FIG. 1A depicts exemplary image analysis methods for measuring facial wrinkles in a subject.

FIG. 1B depicts exemplary image analysis methods for measuring deep layer spots in a subject.

FIG. 1C depicts exemplary image analysis methods for measuring pores in a subject.

FIG. 2 depicts a graphical correlation of facial wrinkling with age.

FIG. 3 depicts a graphical correlation of facial deep layer spots with age.

FIG. 4 depicts a graphical correlation of facial visible pores with age.

FIG. 5 depicts an exemplary skin color measurement method.

FIG. 6 depicts a graphical correlation of facial skin tone (ITA°) with age.

FIG. 7 depicts a graphical correlation of facial skin yellowness with age.

FIG. 8 depicts an exemplary method of measuring skin color and tone evenness.

FIG. 9 depicts a graphical correlation of facial skin color unevenness with age.

FIG. 10 depicts exemplary facial skin images for measuring facial skin translucency.

FIGS. 11A-11C depict an exemplary method of measuring facial skin translucency.

FIGS. 12A-12C depict an exemplary method of measuring facial skin translucency.

FIG. 13 depicts a graphical correlation of facial skin translucency with age.

FIG. 14 depicts exemplary facial skin images for measuring surface homogeneity.

FIG. 15 depicts a graphical correlation of facial skin surface homogeneity with age.

FIG. 16 depicts a graphical correlation of facial skin redness with age.

FIG. 17 depicts a graphical correlation of age with Skin Youthfulness Index.

FIG. 18 depicts a graphical distribution of Skin Youthfulness Index in all age groups in Asian female subject under investigation.

FIGS. 19A-19B depict a before (FIG. 19A) and after (FIG. 19B) images of a subject undergoing a skin treatment.

FIGS. 20A-20B depicts before (FIG. 20A) and after (FIG. 20B) images of a subject undergoing a skin treatment.

FIGS. 21A-21B depicts a before (FIG. 21A) and after (FIG. 21B) images of a subject undergoing a skin treatment.

FIG. 22 depicts a graphical correlation of age with Skin Youthfulness Index.

FIG. 23 depicts a graphical Skin Youthfulness Index Distribution among various age groups.

FIG. 24 depicts a graphical correlation of Skin Youthfulness Index to age illustrating the difference before and after product use.

FIG. 25 depicts a graphical correlation of Skin Youthfulness Index with age.

FIG. 26 depicts examples of smart devices.

FIG. 27 depicts a method for measuring an improvement of skin characteristics following a treatment in connection with a smart device.

FIG. 28 shows a flowchart for calculating weight factor for each parameter.

FIGS. 29A-29J show the average values of each measured visual parameter plotted against the subjects' chronological age.

FIG. 30 is a graph showing a correlation between the actual age and the predicted age of each group with a RSS=215.32.

FIG. 31 is a graph showing an optimal parameter combination.

FIG. 32 is a graph showing a correlation between skin youthfulness index and age.

FIG. 33 is a graph showing the normalized SYI distributions for each of the nine age groups.

FIG. 34 is a graph showing a correlation plot of predicted ages to actual ages of the nine test groups.

### DETAILED DESCRIPTION

Sophisticated facial imaging systems make it possible to objectively measure visual properties of the skin using image analysis apparatus and methods. For example, the present inventors have been associated with a program that has collected images from more than 21,000 people around the world covering a wide range of age and ethnicity in both genders. Such databases allow for analyzing multiple visual parameters of facial skin and correlating them with age accurately and reliably.

As such, the present invention relates to improved methods of correlating skin parameters with age by establishing a Skin Youthfulness Index (SYI), which incorporates visual skin parameters objectively measured from facial images of human subjects.

The skin youthfulness index represents a unique approach to determine skin youthfulness and/or an apparent skin age of a person and/or to determine a degree of improvement, if any, in the apparent facial skin appearance of a person following skin treatment. The SYI may be determined by combining multiple objectively measured visual skin parameters into one linear, composite function to correlate the parameters with age of a subject. The SYI value represents the properties of the skin at various ages, i.e., younger skin correlates to a higher value of SYI and older skin correlates to a lower value of SYI. Such correlations find use, for example, in measuring the efficacy of a skin treatment on a human skin to determine the apparent age change of the person's skin as a result of the skin treatment. Consequently, reliance on subjective parameter measurement is substantially or completely eliminated so that the resulting values are more accurate in assessing skin conditions and characteristics in terms of skin youthfulness for beauty-conscious consumers.

The term “skin” refers to cell layers comprising the integument of a human individual and include the skin on the face, neck, chest, back, torso, arms, axillae, hands, legs, and scalp, and its structural components such as hair, hair follicles, sebaceous glands, apocrine (sweat) glands, fingernails and toenails. Furthermore, the term “skin” as used herein encompasses tissues of the mucous membranes extending from the adjoining skin, such as the mouth and oral cavity, nose and nasal passages, eyes and eyelids, ears and outer ear canals. The term “facial skin” refers to cell layers comprising the skin on the face and its structural components. Although throughout this application facial skin is discussed, any part of human skin may be evaluated and/or correlated with age according to the methods of the present invention.

The term “treatment area” refers to a region of the skin that is to be treated with a skin treatment, including any medical or cosmetic treatment. In a preferred embodiment of this invention, the affected area may be a site for which improvement of a cosmetic nature is sought, and can also include all skin on an individual.

The term “skin youthfulness” refers to a characteristic of skin and relates to the general freshness and vitality characteristic of a young person's skin.

The term “apparent skin age of a subject” refers to an age value determined by correlating the SYI of the subject with a standard reference curve of SYI to age.

The term “objectively measured” refers to methods of analyzing and measuring skin parameters with the use of instruments and imaging methods.

The terms “improvement of skin characteristics” and “improvement of facial skin characteristics” refer to an increase in value of SYI measured following a skin treatment as compared to a value of SYI measured before the treatment.

The term “cosmetic composition” is intended to describe compositions for topical application to human skin, including leave-on and wash-off products.

The term “skin color” is a general term intended to cover human perception of color and includes variations in lightness/darkness and/or variations in hue or skin tone.

“Brightness” is defined in terms of the L* parameter in the L*-a*-b* color space. The greater the L* value, the lighter the skin. The smaller the L* value, the darker the skin, indicating higher melanin content.

“Redness” refers to degree of red color in the skin tone of an individual and is defined as a* value. The higher the a* value, the more red tones are present in the skin.

The term “yellowness” refers to a degree of the yellow color in the skin tone of an individual. Yellowness is defined as a b* value. The higher the b* value, the more yellow tones are present in the skin.

Usually for skin color, a* and b* are greater than zero.

### I. Skin Youthfulness Index

Certain embodiments relate to an SYI having an inverse correlation to human age and describing a skin youthfulness (i.e., an apparent skin age) of a human subject on a 0 to 100 scale with a higher value corresponding to a more youthful appearance (i.e., a younger-looking skin) than the chronological age of the subject.

In certain embodiments, the SYI comprises:

SYI=10(10+(0.081 ln T−0.627 ln Wr−0.174 ln P−0.056 ln b*−0.062 ln U) Equation 11

wherein:

T corresponds to translucency index,

Wr corresponds to wrinkle score,

P corresponds to pore score,

b* corresponds to yellowness, and

U corresponds to color unevenness.

In certain other embodiments, the SYI is determined by:

$$SYI = N {\sum }_{i = 1}^{9} {W}_{i} {V}_{i}^{j} ; \qquad ( Equation 1 )$$

wherein:

W corresponds to a Weight Factor (W);

V corresponds to a value of an objectively measured visual skin parameter (normalized);

i corresponds to a specific type of the measured visual skin parameter:

1=wrinkles, 2=deep layer spots, 3=pores, 4=translucency, 5=skin redness, 6=skin yellowness, 7=Individual Typology Angle (ITA°), 8=unevenness of skin tone, 9=surface textural parameter (entropy);

j=1 for a positive correlation;

j=−1 for a negative correlation; and

N is a factor to target SYI in a 0-100 value.

The Weight Factor (W) is determined by normalizing a value of an Impact Factor (IF) to a 0-1 scale.

In certain embodiments the IF is determined by:

IF=SF×MIF (Equation 2);

wherein:

SF corresponds to a Significance Factor (SF), wherein a SF value is determined for each individual measured visual skin parameter having a Pearson correlation coefficient greater than a critical value, and where the SF value for each individual measured visual skin parameter is determined by:

SF=r²×r² (Equation 3);

where r² is the coefficient of determination, and

MIF corresponds to Maximum Impact Factor (MIF) and is determined for each individual measured visual skin parameter by:

MIF=(Maximum−Minimum)/Average (Equation 4);

wherein:

Maximum corresponds to a maximum value measured for each individual visual skin parameter;

Minimum corresponds to a minimum value measured for each individual visual skin parameter; and

Average corresponds to an average value for each individual measured visual skin parameter.

### II. Methods of Determining Skin Youthfulness Index

In certain embodiments, the present invention relates to a method of determining an SYI of a subject.

The method of the present invention includes measuring at least one and maximum of nine visual skin parameters of the subject, normalizing data from the measured visual skin parameters, calculating the Weight Factor for the measured visual skin parameters, and calculating the SYI of the subject. The SYI may be calculated as described above in Equation 1.

In certain other embodiments, at least two but a maximum of nine visual parameters are measured.

In certain alternative embodiments, one visual parameter is measured; alternatively, two visual parameters are measured; alternatively, three visual parameters are measured; alternatively, four visual parameters are measured; alternatively, five visual parameters are measured; alternatively, six visual parameters are measured; alternatively, seven visual parameters are measured; alternatively, eight visual parameters are measured; and alternatively, nine visual parameters are measured.

The step of measuring visual skin parameter(s) of the subject may include accessing a multiplicity of photographic images of human faces with each image having associated with it a chronological age of the human whose likeness it captures and analyzing the images for the visual skin parameters.

Photographic images include any conventional media capable of capturing the image of a human subject, such as digital or analog electronic cameras, including mobile device cameras, as well as, conventional film based photographic techniques. The images may be created with any type of light that is capable of capturing a chronological age related feature of a human subject, including, for example, standard, flat, UV or narrow-band blue light, cross polarized, and parallel polarized light. The images may be captured and stored in electronic form or in the form of printed photographic images or in any other form in which the images may be accurately visualized. Preferably, the multiplicity of images is obtained from a multiplicity of humans of racial background and gender, and across a range of ages. Also, preferably, the multiplicity of humans is of sufficient number to provide statistical significance.

For example, in certain embodiments, the measuring of the visual skin parameters may be conducted from at least one image taken of a subject. Preferably, multiple images are taken of the subject. The image(s) may be a photograph taken by a camera used to acquire images of subjects. In certain embodiments, a subject may be photographed under five different lighting conditions, such as standard, flat, UV or narrow-band blue light, cross polarized, and parallel polarized.

The images are then assessed for a measured skin parameter that changes as a human ages. As discussed above, the visual skin parameters preferably include: wrinkles, deep layer spots, pores, translucency, skin redness, skin yellowness, Individual Typology Angle (ITA°), unevenness of skin tone, and surface textural parameter (entropy). The wrinkles may include eye corner wrinkles (crow's feet), cheek wrinkles, forehead wrinkles, under-eye wrinkles, nasolabial folds, frown lines, or the like, or a combination thereof. Other visual parameters, such as contrast between facial features (such as eyes, lips, etc.) and facial skin, lip line smoothness, facial contour, skin sagginess, under-eye puffiness and dark circles may also be used.

The measuring of the visual skin parameters is performed by objective measurement, including measurement by an instrument using methods known in the art.

In certain embodiments, the measuring step includes measuring anywhere from one to nine visual skin parameters; however, preferably, five visual skin parameters are measured selected from: wrinkles, deep layer spots, pores, translucency, skin redness, skin yellowness, Individual Typology Angle (ITA°), unevenness of skin tone, and surface textural parameter (entropy) of the subject, may be measured.

The hardware for the imaging and analysis of photographs can include any suitable image analysis hardware. For example, the hardware for the imaging and analysis of photographs may include the VISIA-CR (Canfield, U.S.A.), which captures a set of images from a person under five different lighting conditions, such as flat, standard, UV or narrow-band blue light, cross polarized, and parallel polarized. Using image analysis software, the visual properties of facial skin, such as wrinkles, pores and subsurface pigmented spots, etc. can be quantified and/or scored from the set of captured images according to the known methods (Whitehead et al. 2010). In addition, an image analysis methods developed described herein may be used to quantify other properties of the facial skin, such as skin color parameters, lightness of skin tone (in terms of ITA°), evenness of skin tone, skin surface texture parameters using the commonly accepted statistical method of Gray Level Co-occurrence Matrix (GLCM), as well as skin translucency parameter, which is defined as the amount of subsurface reflection over the total reflection using information from the cross and parallel polarized images (Matsubara, 2012).

Next, linearity of each measured visual parameter with age may be examined. The parameter scores are then normalized to show values within 0-100 scale and their linearity with age is examined. Any parameter with a Pearson correlation coefficient greater than the critical value is accepted for use in calculations. The critical value of this linearity test may be obtained from statistical analysis of the data set.

Next, a Significance Factor (SF) for each of the measured visual parameters is calculated. Any parameter with a Pearson correlation coefficient greater than the critical value, has a SF of:

SF=r²×r² (Equation 3).

The better the linearity of the measured visual parameter, the greater contribution this parameter will have on the overall skin youthfulness index.

Maximum Impact Factor(s) (MIF) may then be determined for each of the measured visual parameters since each parameter affect the skin youthfulness differently.

The MIF may be determined by:

MIF=(Maximum−Minimum)/Average (Equation 4).

As demonstrated below, this factor effectively defines the different levels of impact of the various measured skin parameters.

Next, the Impact Factor (IF) may be determined for each of the measured visual parameters by:

IF=SF×MIF (Equation 2).

Next, the W and SYI are calculated for the subject.

Specifically, the W may be calculated for each of the measured visual parameters of the skin by normalizing the Impact Factors in a 0-1 scale. The SYI may then be determined according to Equation 1 above.

The SYI of the subject can then be compared to a standard reference curve to determine an apparent age of the subject.

### III. Standard Reference Curve

To establish an age correlation with the visual skin properties and to establish a standard reference curve, images of people at exact and almost exact chronological ages are utilized (e.g., 20, 25, 30, 35, 40, 45, 50, 54-55, and 59-61 years old). These selections of individuals at exact and almost exact chronological ages or age ranges are more advantageous than other means traditionally employed in the industry, where, for example, 30 subjects may be selected in a wide age range of 20-29 years to represent people in their 20s. The advantage of having a standard curve created based on the exact or almost exact chronological ages or age ranges is that the resulting standard reference curve will provide a more accurate illustration of the facial skin characteristics of people at those exact and almost exact chronological ages.

The images are then analyzed to calculate SYI of the subjects as described above. Statistical analysis methods may be used to examine the correlation of every visual facial skin parameter for its significance, as well as its contribution to the comprehensive SYI may be calculated. The SYI averages for the exact ages or almost exact ages are correlated to chronological age to create a standard reference curve.

Once generated, the reference curve may be used, for example, to determine the apparent age or facial skin youthfulness of a subject.

In certain embodiments, the standard reference curve may be used for the before- and after-skin treatment correlations of skin youthfulness of an individual to chronological age. For example, an untreated subject human face is photographed and the skin parameters are measured to determine a before the skin treatment SYI (SYI_{b}) of the subject according to the same methods used to determine the SYI of a subject described above and according to Equation 1. The SYI_{b} may then be correlated with the age standard reference curve to determine a before-the treatment apparent age value of the subject. In certain embodiments, the before-the treatment age value may be lower than the actual chronological age of the subject; in other embodiments, the before-the treatment age value may be higher than the actual chronological age of the subject. If so desired, the subject is then treated with a skin treatment. The term “skin treatment” refers to any skin care regimen that is intended to have a noticeable effect on human facial appearance and includes, for example, nutritional programs, the use of topical skin care products, the use of skin care devices, oral dosage forms, massage therapy, radiation treatments, and the like, and combinations thereof. For example, the skin treatment may be a cosmetic skin treatment. Alternatively, the skin treatment may be a medical skin treatment. A “cosmetic treatment” is a non-medical procedure to help the health and appearance of the facial skin. Cosmetic treatments may be topical treatments or procedures that may be performed at individual's home, cosmetic/beauty salons, spas (e.g., destination or day spa), cosmetic schools, or at a doctor's office. Examples of cosmetic facial skin treatments include facials; chemical (glycolic acid or trichloroacetic acid) and physical peels (dermabrasion or microdermabrasion); IPL/Photorejuvenation; laser skin resurfacing; and others. Other examples of cosmetic treatments include treatments with lotions or creams that include cosmetic compositions, including e.g., anti-aging agents, such as Retin A, resveratrol, allantoin, vitamin C, vitamin E, and peptides.

An example of a medical treatment includes plastic surgery to make the skin look younger or more youthful and may include: partial or full-face lifts, laser treatments, skin resurfacing, wrinkle treatments, such as treatments with neurotoxins (Botox™, Dysport™ and Xeomin™), Injectable Fillers, Hyaluronic Acids (Juvederm™, Restylane™ and others), Hydroxyapatitie microspheres (Radiesse™), Poly-L-Lactic Acid (Sculptra™), Polymethylmethacrylate microspheres/collagen (Artefill™), and fat grafting.

Other cosmetic and medical treatments will be known to those skilled in the art.

Examples of skin care products include: a lotion, a cream, a cleanser, a scrub, a gel, a liquid, a powder, a toner, an astringent, a masque, a serum, and combinations thereof. Preferably, the skin care product is an anti-aging topical skin treatment product.

In some embodiments, the skin treatment is a treatment intended to improve the appearance of a subject, e.g., a treatment intended to make the subject look more youthful and healthy as compared to the chronological age of the subject. As discussed above, one example of such treatment is treatment with a care product that is an anti-aging skin treatment product.

After the subject's face has been treated with the skin treatment for a given length of time (e.g., days, weeks, years; preferably at least 1 week, 2 weeks, 3 weeks, 4 weeks, or longer, etc.), the treated subject human face is photographed again and the skin parameters are measured to determine an after the skin treatment SYI (SYIₐ) of the subject according to the same methods used to determine SYI_{b} of a subject described above and according to Equation 1. The SYIₐ is then correlated with the age standard reference curve to determine an after the treatment apparent age value of the subject.

The difference between the before the treatment apparent age value and the after the treatment apparent age value to measure improvement, if any, of facial skin characteristics following the treatment may then be determined. The apparent age difference indicates the difference in the apparent age values and may be used as a measure of the efficacy of the skin treatment. This parameter may be useful in communicating the effectiveness of the skin treatment.

### IV. Applications and Uses of SYI

In certain embodiments, the present invention relates to a method of determining an apparent age of a subject. The method includes the steps of (i) measuring one to nine visual skin parameters of the subject; (ii) normalizing data from each of the measured visual skin parameters; (iii) calculating a Weight Factor (W) for the measured visual skin parameter; (iv) calculating a SYI of the subject; and (v) correlating the SYI of the subject to a standard reference curve to determine the apparent age of the subject.

The measuring step may include measuring anywhere from one to nine visual parameters of the subject, but preferably, measuring five visual parameters of the subject.

Similarly as described above in connection with the method of determining the SYI, the measuring step may be conducted from an image of the subject.

In certain embodiments, the method may further include photographing the subject under five different lighting conditions, such as standard, flat, UV or narrow-band blue light, cross polarized, and parallel polarized.

In certain embodiments, in the method, the visual skin parameters that are measured may be wrinkles, deep layer spots, pores, translucency, skin redness, skin yellowness, Individual Typology Angle (ITA°), unevenness of skin tone, and/or surface textural parameters (e.g. entropy).

In certain other embodiments, the measuring step may include measuring anywhere from one to nine visual skin parameters, such as wrinkles, deep layer spots, pores, translucency, skin redness, skin yellowness, Individual Typology Angle (ITA°), unevenness of skin tone, and surface textural parameter (entropy) of the subject. Preferably, the nine parameters are measured; more preferably five parameters are measured including wrinkle score (Wr), pore score (P), translucency index (T), skin yellowness (b*), and unevenness of skin tone (U).

In a method according to the present invention, the SYI may be determined by Equation 1. The SYI of the subject can then be compared to a standard reference curve to determine an apparent age of the subject.

Alternatively, in the method according to the present invention, the SYI may be determined by Equation 11. The SYI of the subject can then be compared to a standard reference curve to determine an apparent age of the subject.

In certain embodiments, the present invention includes a method for measuring an improvement or evaluating the effectiveness or a skin treatment on facial skin characteristics.

The method includes:

(I) measuring visual skin parameters: wrinkle score (Wr), pore score (P), translucency index (T), skin yellowness (b*), and unevenness of skin tone (U) before and after the treatment;

(II) determining a before-the treatment skin youthfulness index (SYI_{b}) value, wherein SYI_{b} is calculated:

SYI_{b}=10(10+(0.081 ln T_{b}−0.0627 ln Wrb_{b}−0.174 ln P_{b}−0.056 ln b*_{b}−0.062 ln U_{b}) (Equation 13)

(III) determining an after-the treatment skin youthfulness index (SYIₐ) value, wherein the SYIₐ is calculated:

SYIₐ=10(10+(0.081 ln Tₐ−0.0627 ln Wrbₐ−0.174 ln Pₐ−0.056 ln b*ₐ−0.062 ln Uₐ) (Equation 14)

(IV) correlating the SYI_{b} value to an age standard reference curve to determine a before-the treatment age value;

(V) correlating the SYIₐ value to the age standard reference curve to determine an after-the treatment age value; and

(VI) determining the difference between the before-the treatment age value and the after-the treatment age value to measure improvement, if any, of facial skin characteristics following the treatment.

The treatment may be a cosmetic treatment and/or a medical treatment.

In another alternative embodiment, the method includes the steps of (i) measuring visual skin parameters: wrinkles, deep layer spots, pores, translucency, skin redness, skin yellowness, Individual Typology Angle (ITA°), unevenness of skin tone, and surface textural parameter (entropy) before and after the treatment; (ii) determining a before-the-treatment skin youthfulness index (SYI_{b}) value; (iii) determining an after-the-treatment skin youthfulness index (SYIₐ) value; (iv) correlating the SYI_{b} value to an age standard reference curve to determine a before-the-treatment age value; (v) correlating the SYIₐ value to the age standard reference curve to determine an after-the-treatment age value; and (vi) determining the difference between the before-the-treatment age value and the after-the-treatment age value to measure the improvement, if any, of facial skin characteristics following the treatment.

In the method, the measuring step may be conducted from an image.

In certain embodiments, the method may further include photographing a subject under five different lighting conditions: standard, flat, UV or narrow-band blue light, cross polarized, and parallel polarized.

Similarly, as described in connection with the methods above, the treatment may be a cosmetic treatment or a medical treatment. Examples of cosmetic and medical treatments were provided above.

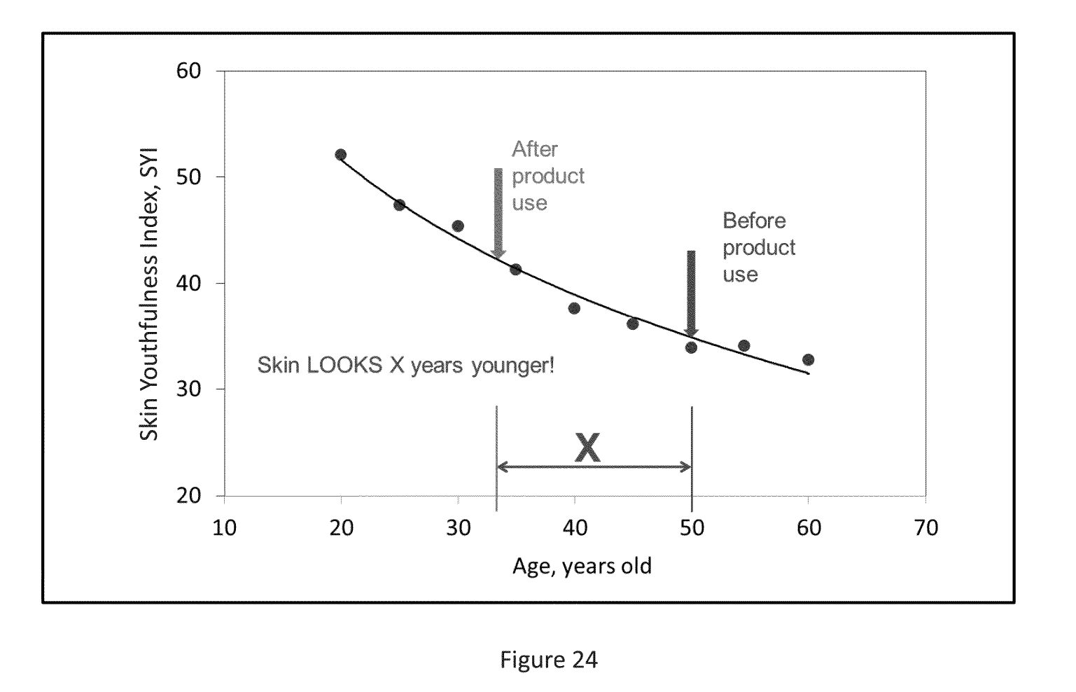

Providing an age/SYI correlation allows one to describe skin age in terms of skin youthfulness, as an index that shows positive aspects of skin and a concept that can be easily received by beauty-conscious consumers. For example, on average, an individual having younger skin (e.g., an individual with a chronological age of 20 years) may exhibit a skin youthfulness index of 52, while, on average, an individual having older or more mature skin (e.g., an individual with a chronological age of 45 years) may exhibit a SYI index of 36. Improving the value of SYI from 36 to a higher number may indicate the consumer achieved a younger-looking skin. Also, the efficacy of skin care products and/or treatments may therefore be evaluated. This concept is illustrated in FIG. 24.

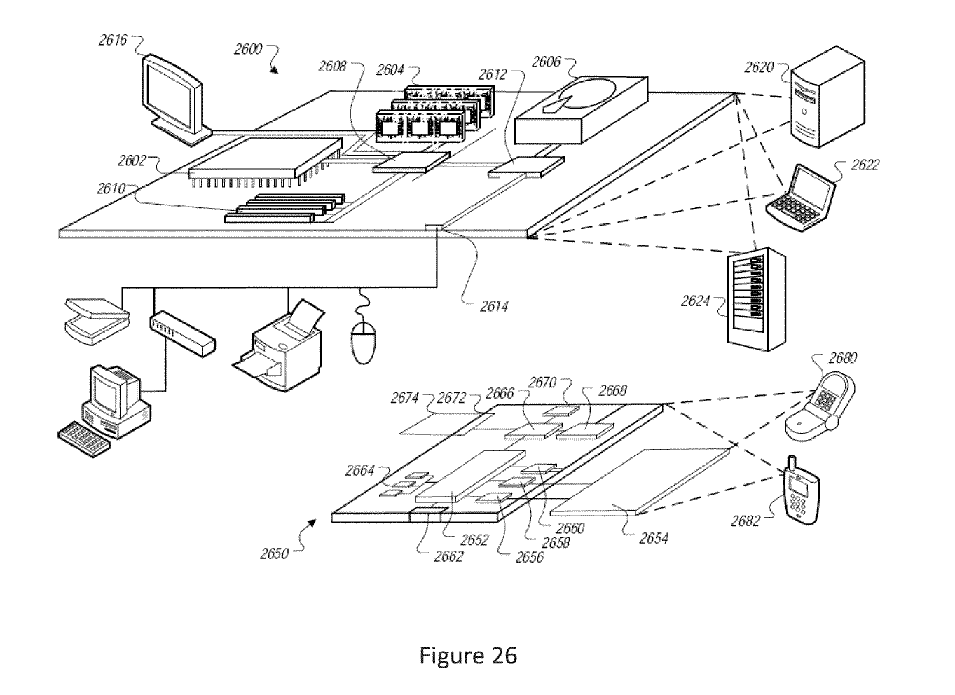

In certain embodiments, the mathematical algorithm for SYI may be incorporated into a computer software, or mobile device applications, such as, for example, in one of the smart devices discussed below in connection with FIG. 26. For example, pictures taken from a smart phone may be analyzed and processed to generate a SYI of an individual by the same individual or another individual (e.g., a service, a physician, etc.).

In other embodiments, the skin youthfulness index may be used to design technology strategies for anti-aging formulations. For example, with the understanding that visual skin parameters, such as wrinkles, have the most significant effect when determining the skin youthfulness index, one may be able to focus efforts on incorporating efficacious wrinkle reduction technology into an anti-aging formulation to increase the probability of achieving success. In addition, skin yellowness reduction may also be considered when designing skin formulations. As such, it may be beneficial to incorporate multiple anti-aging strategies into a formulation when formulating anti-aging or youthfulness-promoting skin compositions or products.

### V. Smart Device

In certain embodiments, the systems and methods disclosed above may be for use with a smart device. A smart device may be implemented in the form of one or more computing devices.

FIG. 26 illustrates examples of computing devices 2600, 2650 that may be used to implement the systems and methods described in this document. Computing device 2600 is intended to represent various forms of digital computers, such as laptops, desktops, workstations, personal digital assistants, servers, blade servers, mainframes, and other appropriate computers. Computing device 2650 is intended to represent various forms of mobile devices, such as personal digital assistants, cellular telephones, smartphones, and other similar computing devices. The components shown here, their connections and relationships, and their functions, are meant to be exemplary only, and are not meant to limit implementations of the inventions described and/or claimed in this document.

Computing device 2600 includes a processor 2602, memory 2604, a storage device 2606, a high-speed interface 2608 connecting to memory 2604 and high-speed expansion ports 2610, and a low speed interface 2612 connecting to low speed bus 2614 and storage device 2606. Each of the components 2602, 2604, 2606, 2608, 2610, and 2612, are interconnected using various busses, and may be mounted on a common motherboard or in other manners as appropriate. The processor 2602 can process instructions for execution within the computing device 2600, including instructions stored in the memory 2604 or on the storage device 2606 to display graphical information for a GUI on an external input/output device, such as display 2616 coupled to high speed interface 2608. In other implementations, multiple processors and/or multiple buses may be used, as appropriate, along with multiple memories and types of memory. Also, multiple computing devices 2600 may be connected, with each device providing portions of the necessary operations (e.g., as a server bank, a group of blade servers, or a multi-processor system).

The memory 2604 stores information within the computing device 2600. In one implementation, the memory 2604 is a computer-readable medium. In one implementation, the memory 2604 is a volatile memory unit or units. In another implementation, the memory 2604 is a non-volatile memory unit or units.

The storage device 2606 is capable of providing mass storage for the computing device 2600. In one implementation, the storage device 2606 is a computer-readable medium. In various different implementations, the storage device 2606 may be a floppy disk device, a hard disk device, an optical disk device, or a tape device, a flash memory or other similar solid state memory device, or an array of devices, including devices in a storage area network or other configurations. In one implementation, a computer program product is tangibly embodied in an information carrier. The computer program product contains instructions that, when executed, perform one or more methods, such as those described above. The information carrier is a computer- or machine-readable medium, such as the memory 2604, the storage device 2606, or memory on processor 2602.

The high speed controller 2608 manages bandwidth-intensive operations for the computing device 2600, while the low speed controller 2612 manages lower bandwidth-intensive operations. Such allocation of duties is exemplary only. In one implementation, the high-speed controller 2608 is coupled to memory 2604, display 2616 (e.g., through a graphics processor or accelerator), and to high-speed expansion ports 2610, which may accept various expansion cards (not shown). In the implementation, low-speed controller 2612 is coupled to storage device 2606 and low-speed expansion port 2614. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 2600 may be configured to receive data from and/or transmit data to a camera (not shown). The camera may be embedded in the computing device 2600 or may be externally connected to the computing device 2600. The camera may be of any suitable type, such as an analog or digital camera. In certain embodiments, the camera may be configured to capture images of a subject under different lighting conditions, such as standard, flat, UV or narrow-band blue light, cross polarized, and parallel polarized.

The computing device 2600 may be implemented in a number of different forms, as shown in the figure. For example, it may be implemented as a standard server 2620, or multiple times in a group of such servers. It may also be implemented as part of a rack server system 2624. In addition, it may be implemented in a personal computer such as a laptop computer 2622. Alternatively, components from computing device 2600 may be combined with other components in a mobile device (not shown), such as device 2650. Each of such devices may contain one or more of computing device 2600, 2650, and an entire system may be made up of multiple computing devices 2600, 2650 communicating with each other.

Computing device 2650 includes a processor 2652, memory 2664, an input/output device such as a display 2654, a communication interface 2666, and a transceiver 2668, among other components. The device 2650 may also be provided with a storage device, such as a microdrive or other device, to provide additional storage. Each of the components 2650, 2652, 2664, 2654, 2666, and 2668, are interconnected using various buses, and several of the components may be mounted on a common motherboard or in other manners as appropriate.

The processor 2652 can process instructions for execution within the computing device 2650, including instructions stored in the memory 2664. The processor may also include separate analog and digital processors. The processor may provide, for example, for coordination of the other components of the device 2650, such as control of user interfaces, applications run by device 2650, and wireless communication by device 2650.

Processor 2652 may communicate with a user through control interface 2658 and display interface 2656 coupled to a display 2654. The display 2654 may be, for example, a TFT LCD display or an OLED display, or other appropriate display technology. The display interface 2656 may comprise appropriate circuitry for driving the display 2654 to present graphical and other information to a user. The control interface 2658 may receive commands from a user and convert them for submission to the processor 2652. In addition, an external interface 2662 may be provide in communication with processor 2652, so as to enable near area communication of device 2650 with other devices. External interface 2662 may provide, for example, for wired communication (e.g., via a docking procedure) or for wireless communication (e.g., via Bluetooth or other such technologies).

The memory 2664 stores information within the computing device 2650. In one implementation, the memory 2664 is a computer-readable medium. In one implementation, the memory 2664 is a volatile memory unit or units. In another implementation, the memory 2664 is a non-volatile memory unit or units.

The memory may include for example, flash memory and/or MRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 2664, expansion memory 2674, or memory on processor 2652.

Device 2650 may communicate wirelessly through communication interface 2666, which may include digital signal processing circuitry where necessary. Communication interface 2666 may provide for communications under various modes or protocols, such as GSM voice calls, SMS, EMS, or MMS messaging, CDMA, TDMA, PDC, WCDMA, CDMA2000, LTE, or GPRS, among others. Such communication may occur, for example, through radio-frequency transceiver 2668. In addition, short-range communication may occur, such as using a Bluetooth, WiFi, or other such transceiver (not shown). In addition, GPS receiver module 2670 may provide additional wireless data to device 2650, which may be used as appropriate by applications running on device 2650.

The computing device 2650 may be implemented in a number of different forms, as shown in the figure. For example, it may be implemented as a cellular telephone 2680. It may also be implemented as part of a smartphone 2682, personal digital assistant, or other similar mobile device.

The computing device 2650 may be configured to receive data from and/or transmit data to a camera (not shown). The camera may be embedded in the computing device 2650 or may be externally connected to the computing device 2650. The camera may be of any suitable type, such as an analog or digital camera. In certain embodiments, the camera may be configured to capture images of a subject under different lighting conditions, such as standard, flat, UV or narrow-band blue light, cross polarized, and parallel polarized.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

VI. Exemplary Method for Use with a Smart Device

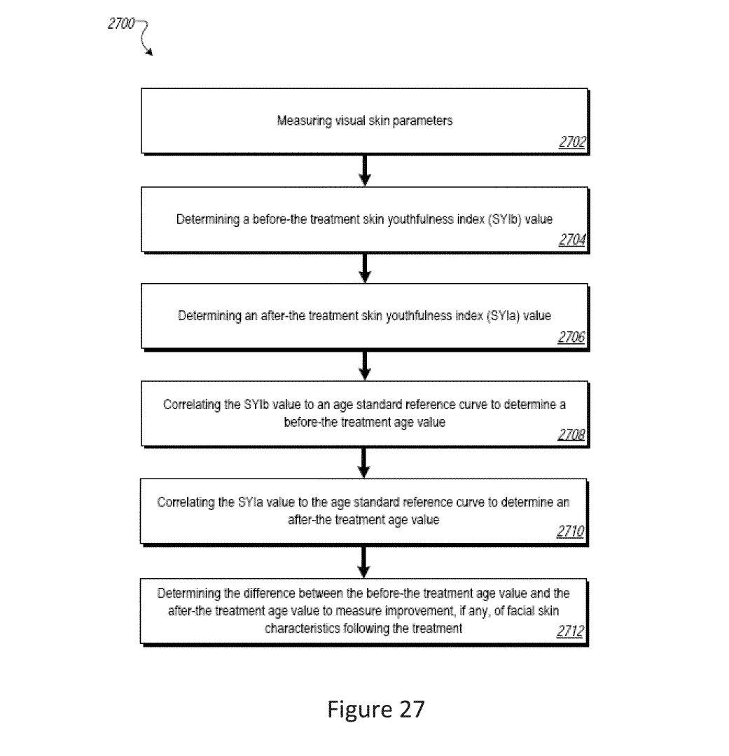

FIG. 27 shows a block diagram 2700 illustrating a method for measuring an improvement of skin characteristics following a treatment, as discussed above, in connection with a smart device, such as the devices 2600, 2650 discussed above.

A smart device may receive photographic images of human faces, with each image having associated with it a chronological age of the human whose likeness it captures and analyzing the images for the visual skin parameters. In some embodiments, the smart device may receive the photographic images from a camera, such as an embedded camera or an externally connected camera. In some embodiments, the smart device may receive the photographic images over a network, such as the Internet, or from another device directly. The smart device may then, by itself or in connection with one or more other devices, use the photographic images to implement steps including those shown in blocks 2702, 2704, 2706, 2708, 2710, and 2712.

Block 2702 includes measuring visual skin parameters: wrinkles, deep layer spots, pores, translucency, skin redness, skin yellowness, Individual Typology Angle (ITA°), unevenness of skin tone, and surface textural parameter (entropy) before and after the treatment.

Block 2704 includes determining a before-the treatment skin youthfulness index (SYI_{b}) value.

Block 2706 includes determining an after-the treatment skin youthfulness index (SYIₐ) value.

Block 2708 includes correlating the SYI_{b} value to an age standard reference curve to determine a before-the treatment age value.

Block 2710 includes correlating the SYIₐ value to the age standard reference curve to determine an after-the treatment age value.

Block 2712 includes determining the difference between the before-the treatment age value and the after-the treatment age value to measure improvement, if any, of facial skin characteristics following the treatment.

### EXAMPLES

### Methods

Standard Reference Curve

To establish an age correlation with visual facial skin properties and to establish a standard reference curve, images of people at exact and almost exact ages were utilized (e.g., 20, 25, 30, 35, 40, 45, 50, 54-55 (to represent people at 54.5 years old), and 59-61 years old (to represent people at 60 years old)). Statistical analysis methods were used to examine the correlation of every visual skin parameter for its significance, and its contribution to the comprehensive Skin Youthfulness Index was calculated.

Nine visual skin parameters were measured, as described below for 9 different chronological age groups shown in Table I.

The subjects were photographed under five different lighting conditions: standard, flat, UV or narrow-band blue light, cross polarized, and parallel polarized. 7525 images of 1505 female subjects were utilized. The images were obtained from four Asian countries (China, Japan, Thailand, and Taiwan) from an image database. The number of subjects examined is indicated in Table I as well.

The images were analyzed using the VISIA-CR (Canfield, U.S.A.) hardware and Amway-exclusive F.A.C.E.S. software for the imaging and analysis of photographs. Using image analysis software, the visual properties of facial skin, such as wrinkles, pores and subsurface pigmented spots were quantified from the set of captured images. Also, image analysis methods described below were used to quantify other properties of the facial skin, such as skin color parameters, lightness of skin tone (in terms of ITA°), evenness of skin tone, skin surface texture parameters using the GLCM method, as well as skin translucency.

Next, the data from each of the measured visual skin parameters was normalized to show values within 0-100 scale and the magnitude of change caused by each parameter was taken into consideration to determine the Weight Factor for each measured visual skin parameter.

1. Measuring Wrinkles, Spot and Pore Scores:

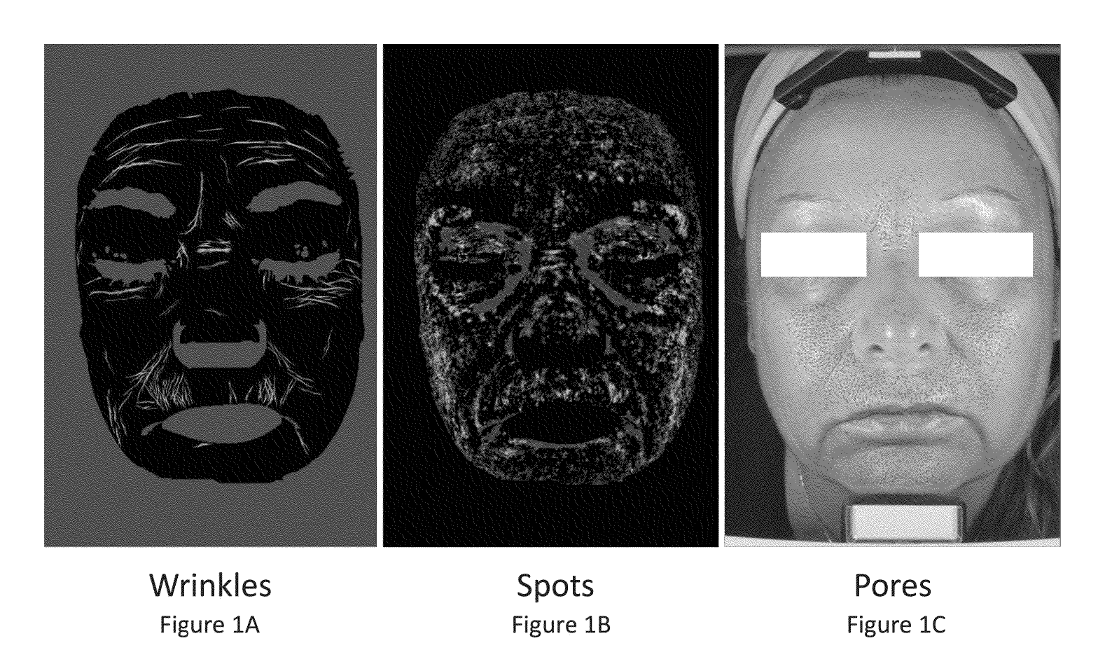

To measure wrinkles, spot and pore scores, a Facial Analysis Computer Evaluation System (F.A.C.E.S.) algorithm was used, as shown in FIGS. 1A-1C.

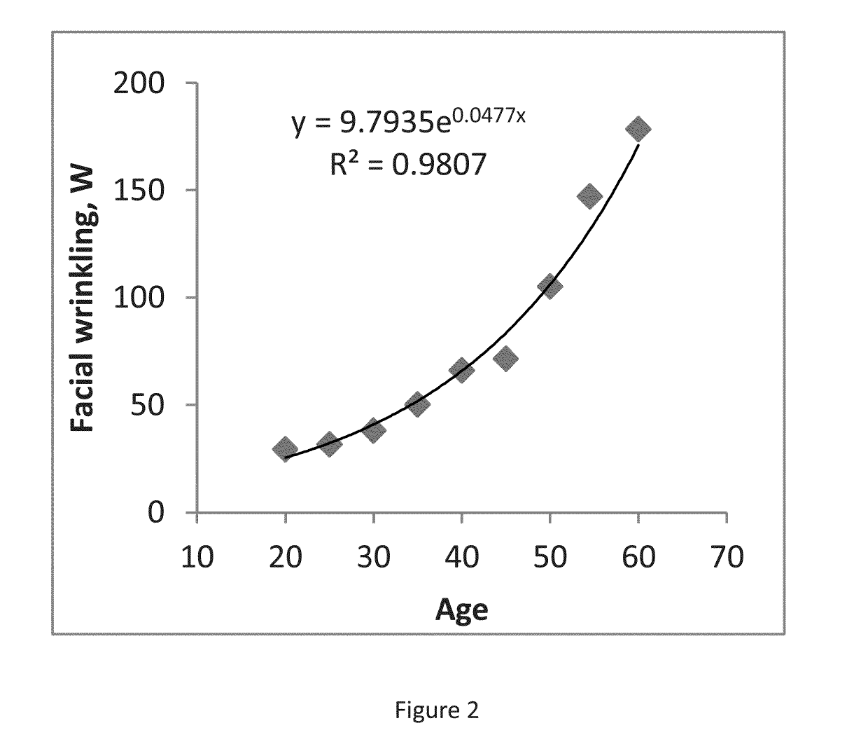

Specifically, as shown in FIG. 2, the total number of wrinkles increased exponentially with increasing age. This may be because the total number of wrinkles reflects the number of wrinkles as well as the severity of wrinkles (a deep wrinkle would be represented by multiple wrinkles lying on top of another wrinkle(s) in one wrinkle location).

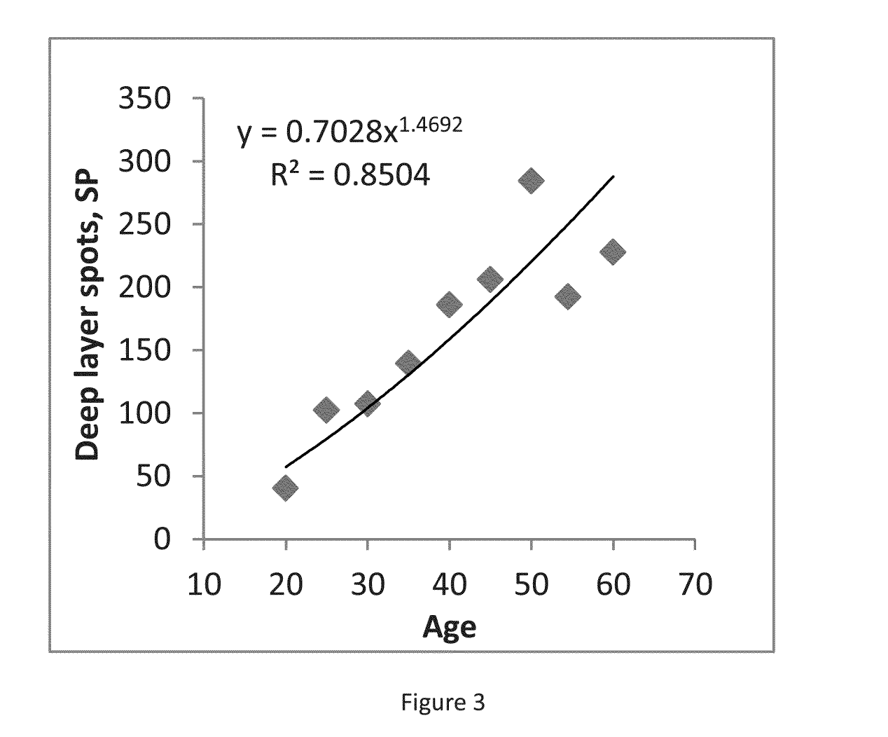

As shown in FIG. 3, the total number of deep layer spots increased with age.

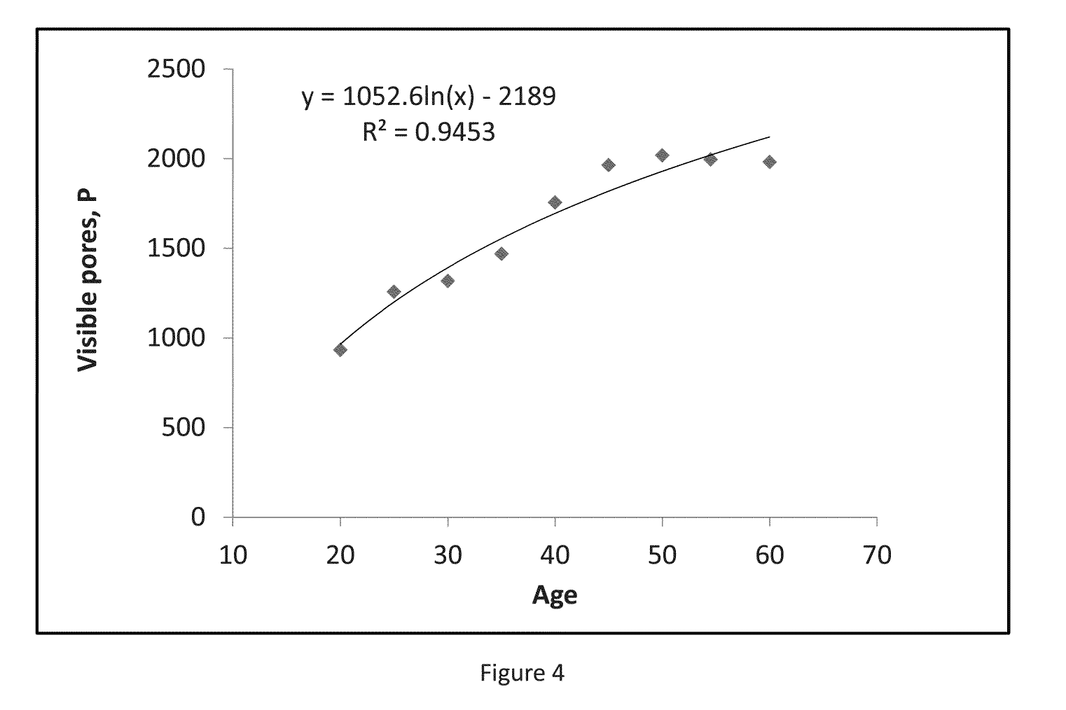

As shown in FIG. 4, the total number of visible pores also increased with age. The changes in color and size with age makes the pores more easily detectable visually and by image analysis; FIG. 1C.

2. Measuring Facial Skin Color and ITA°:

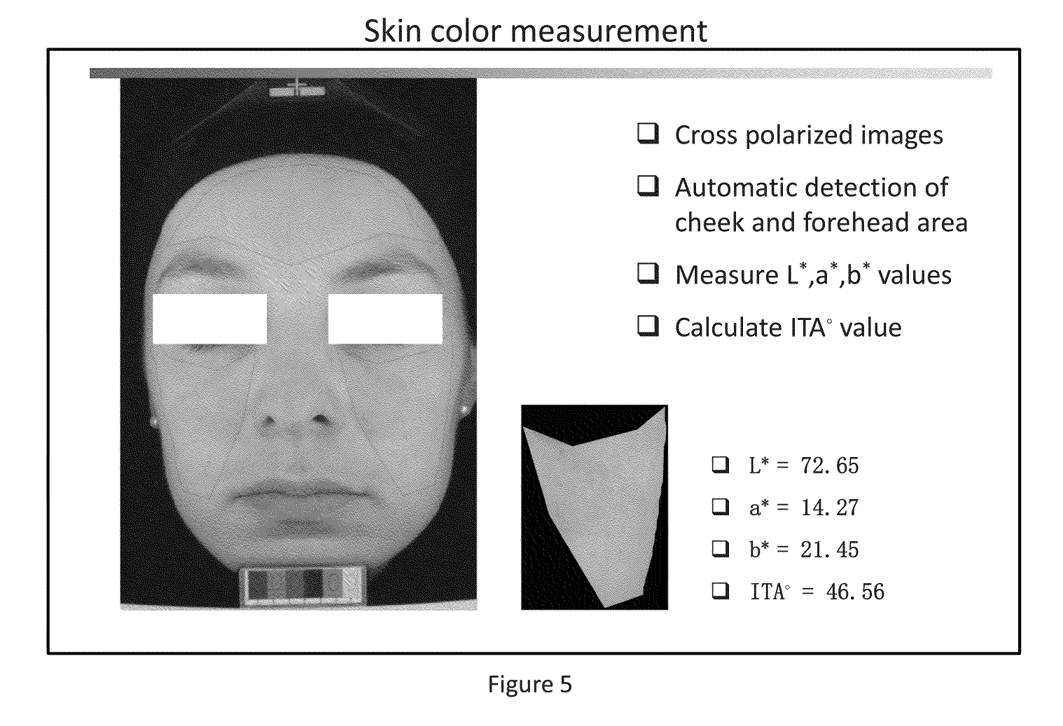

To measure facial skin color and tone uniformity of the studied subjects, an image color correction technology described in U.S. Pat. No. 8,319,857 was used. Specifically, U.S. Pat. No. 8,319,857, which is incorporated herein in its entirety, describes a process that includes measuring color values of a digital color photo, correcting the color deviation of each picture to that of a standard color, and converting the corrected RGB values and generating an output that is useful to L*a*b* values to describe changes in the color properties of the photographed skin (FIG. 5).

Specifically, three areas of face, one on each cheek and one on forehead, were used for this analysis, as highlighted by the areas with yellow borders in FIG. 5. Cross-polarized images were used. The L*a*b* values were average values of those three areas on each image. Typically, during image analysis, each individual image was first color-corrected using the color stripe embedded in the image and a color correction algorithm (U.S. Pat. No. 8,319,857, which is incorporated herein in its entirety). Then, the key features on the face, such as the corners of eyes, mouth, and eyebrows, were detected using an image analysis algorithm, as described below. Those features served as reference points to draw regions of interests (ROI) on the face for color analysis. Average R, G, and B values in each ROI were measured and eventually converted to L*, a* and b* values.

The results were expressed as the skin brightness (L*), redness (a*), and yellowness (b*) values.

The ITA°, which is a measure of lightness of skin tone was then calculated using the following formula:

$$ITA° = \{ arctan ( \frac{{L}^{*} - 50}{{b}^{*}} ) \} \frac{180}{π} \qquad ( Equation 5 )$$

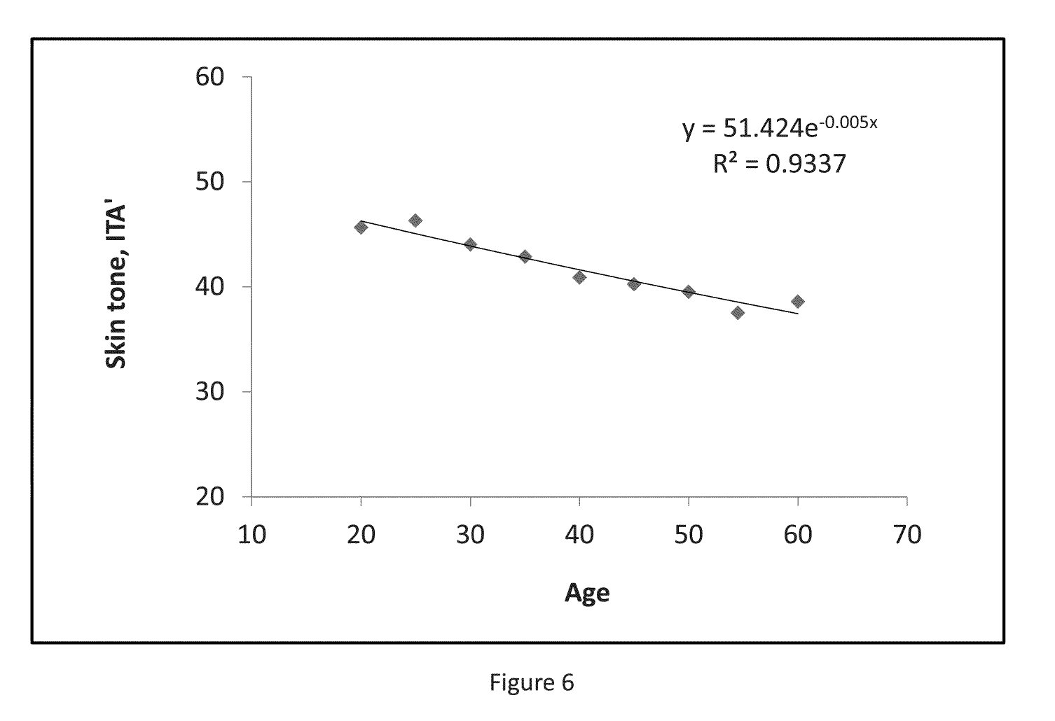

As shown in FIG. 6, the lightness of skin tone decreased with age in roughly linear fashion.

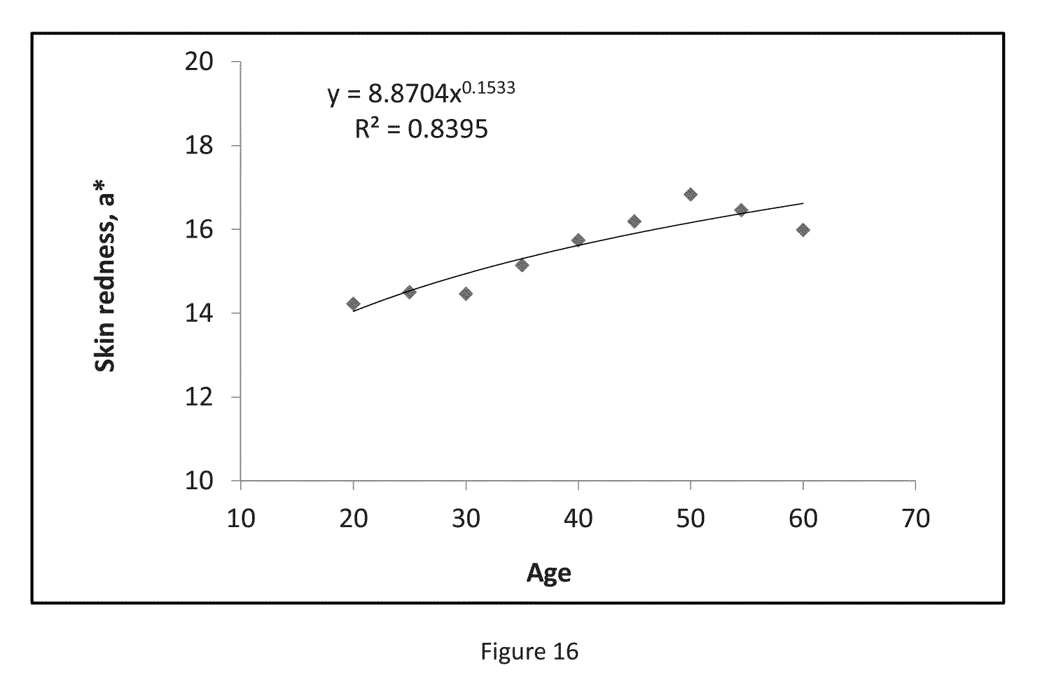

As shown in FIG. 16, skin redness, a*, increased with age.

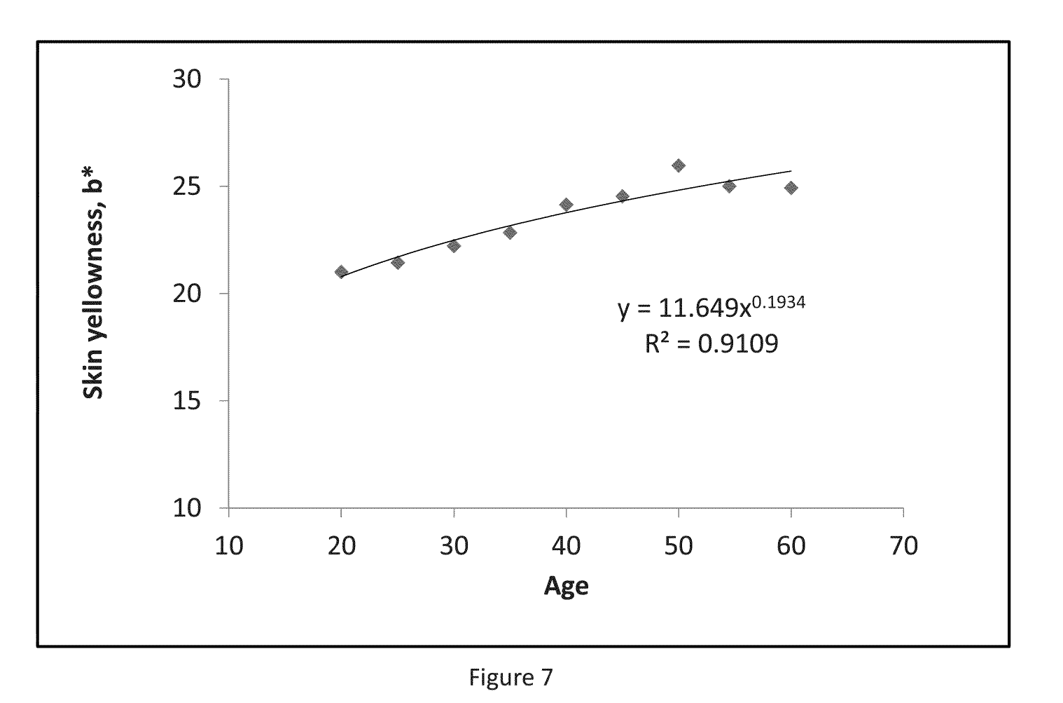

The component of ITA°, skin yellowness, b* increased with age indicating that older people have more yellowish skin tone, as shown in FIG. 7.

3. Measuring Unevenness of Skin Tone:

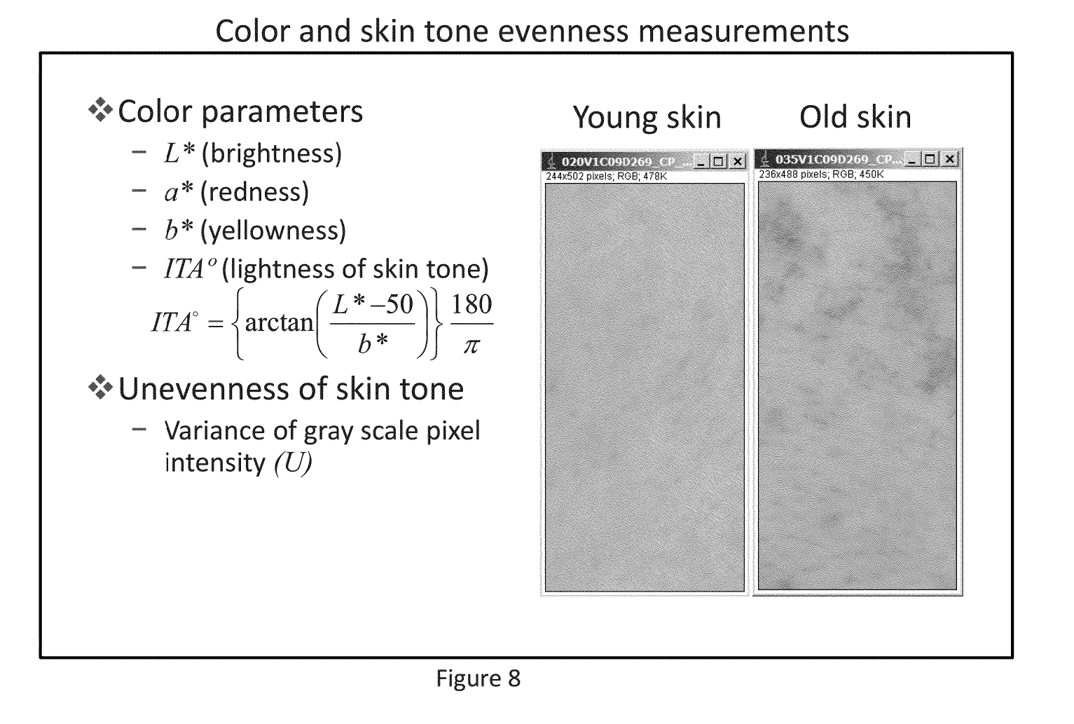

To determine unevenness of skin tone, variance of gray scale pixel intensity (U) was measured (FIG. 8).

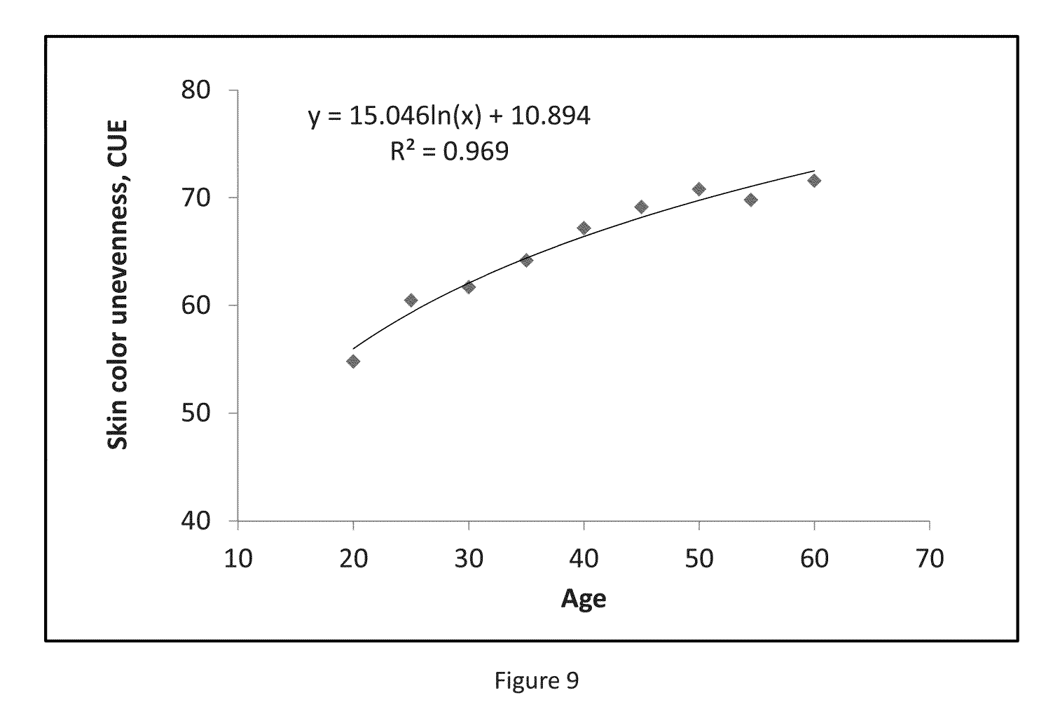

FIG. 9 shows the change in facial skin color unevenness with age; older people have a more uneven skin color tone.

4. Measuring Facial Skin Translucency:

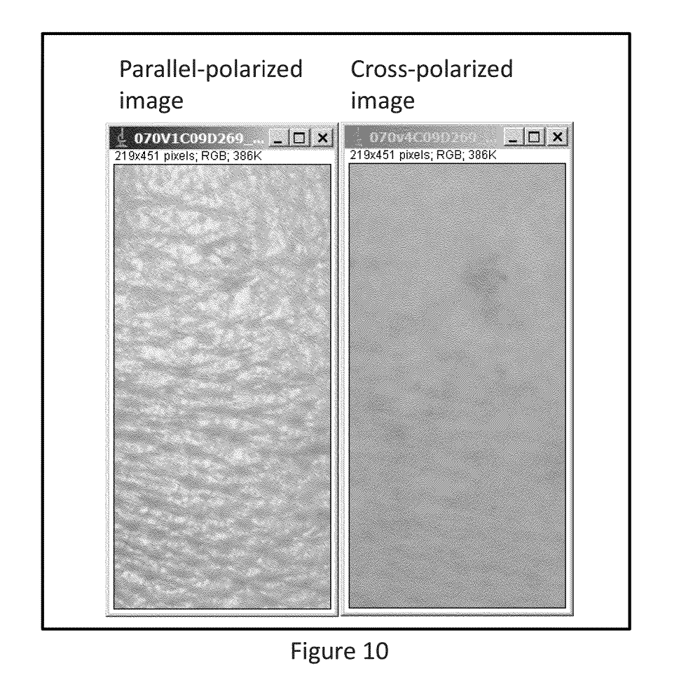

To measure facial skin translucency, with specular and diffuse reflections were calculated from polarized images shown in FIG. 10 (parallel-polarized in the left panel and cross-polarized in the right panel).

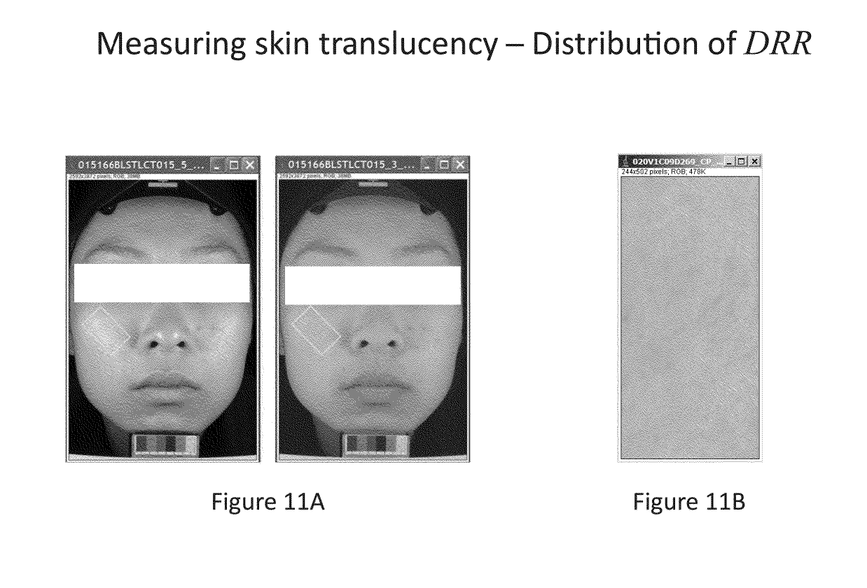

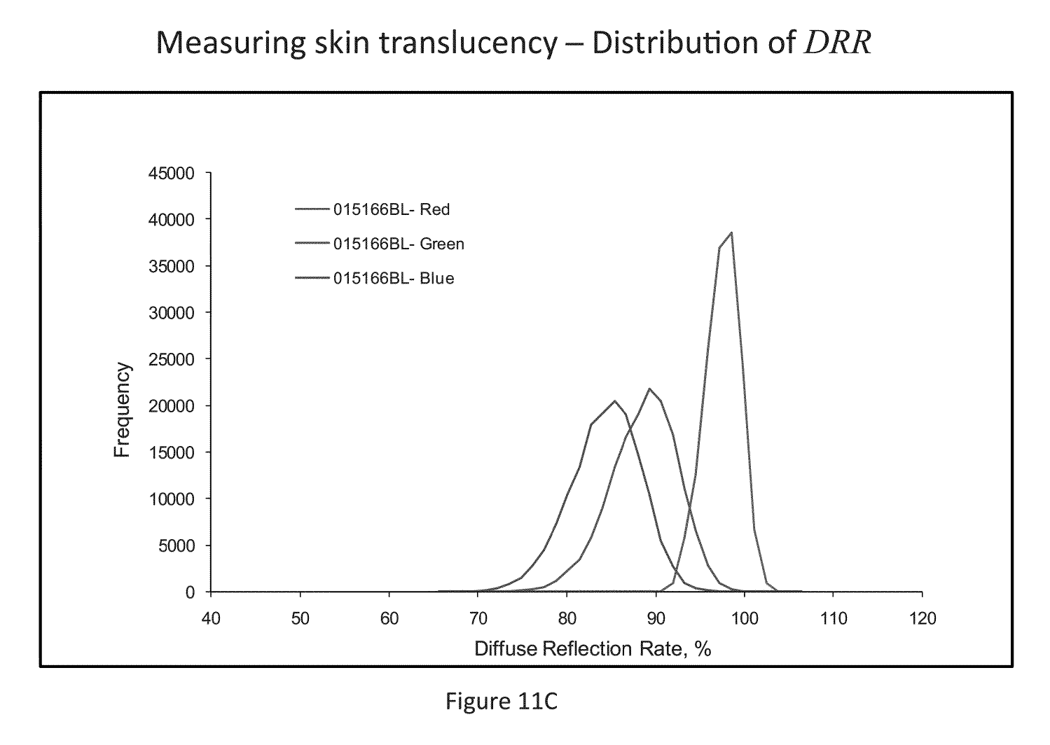

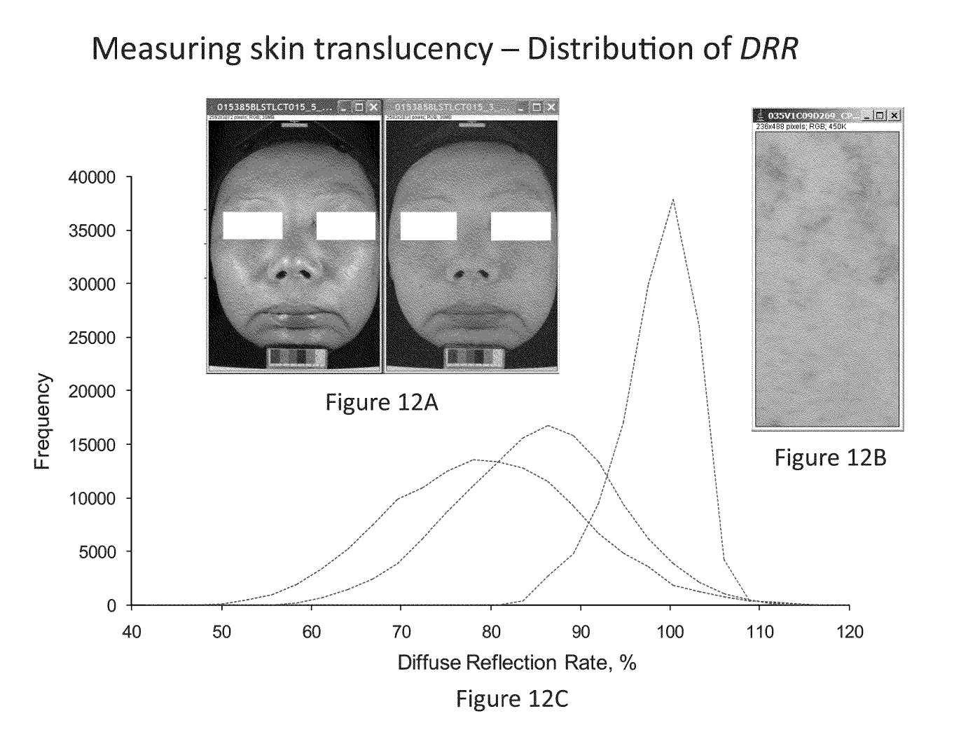

Specifically, first a diffuse reflection rate (DRR) was calculated. Specifically, one area of face (FIGS. 11A and 12A), on each cheek, was used for this analysis, as highlighted by the area with yellow borders in FIGS. 11A and 12A.

The DDR was then calculated using the following formula:

$$\begin{matrix}Diffuse Reflection Rate = \frac{Sublayer reflection}{Total reflection} = \frac{Diffuse}{Diffuse + Specular} & ( Equation 6 ) \\ {DRR}_{pixel , i} = {( \frac{2 {I}_{CP , i}}{{I}_{PP , i} + {I}_{CP , i}} )}_{r , g , b} & ( Equation 7 )\end{matrix}$$

where

I_{CP,i} is the intensity value of i^{th} pixel in a cross-polarized image;

I_{PP,i} is the intensity value of i^{th} pixel in a parallel-polarized image;

r is the red channel of a RGB color image

g is the green channel of a RGB color image

b is the blue channel of a RGB color image

The results are shown in FIGS. 11C and 12C.

The skin translucency was calculated as follows:

$$T = \frac{1}{3} ( \frac{{DRR}_{r}}{{sd}_{DRR , r}} + \frac{{DRR}_{g}}{{sd}_{DRR , g}} + \frac{{DRR}_{b}}{{sd}_{DRR , b }} ) \qquad ( Equation 8 )$$

where

DRRᵣ is Diffuse Reflection Rate for the red channel of a RGB image

DDR_{g} is Diffuse Reflection Rate for the green channel of a RGB image

DRR_{b} is Diffuse Reflection Rate for the blue channel of a RGB image

sd refers to the standard deviation of the pixel intensity in a region of interest (ROI)

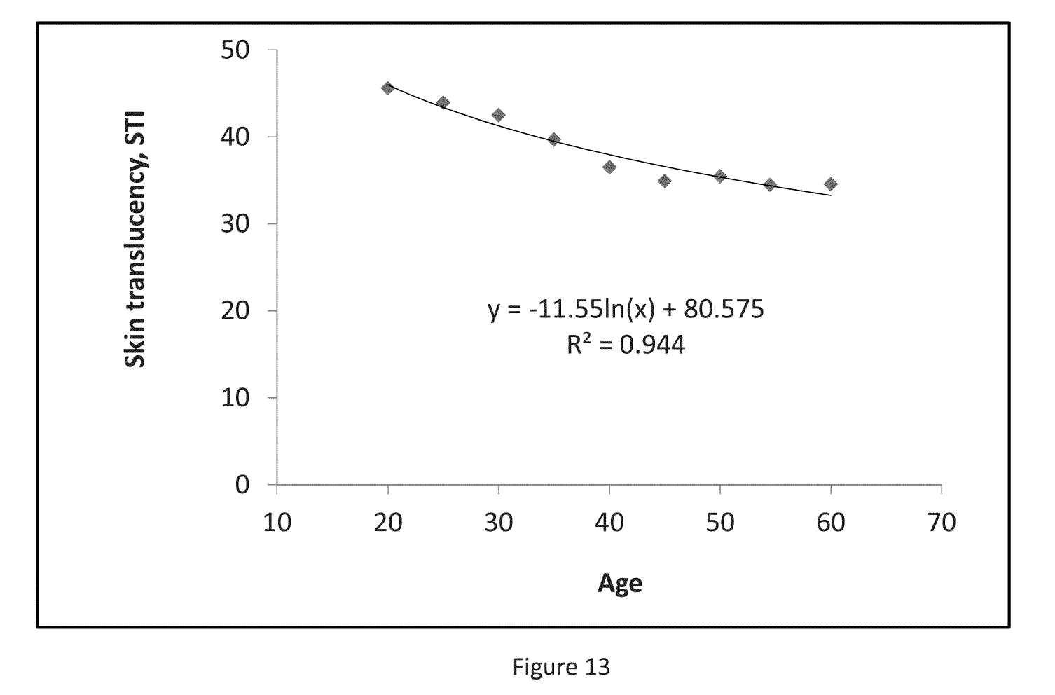

FIG. 13 shows the change in facial skin translucency with age. Specifically, younger people exhibited higher translucency. Younger skin has less specular reflection therefore the colors from the subsurface of skin are more visible.

4. Measuring Skin Surface Texture Parameter:

To measure skin surface texture parameter to describe the textural conditions of the skin, an established image analysis method (GLCM) was used.

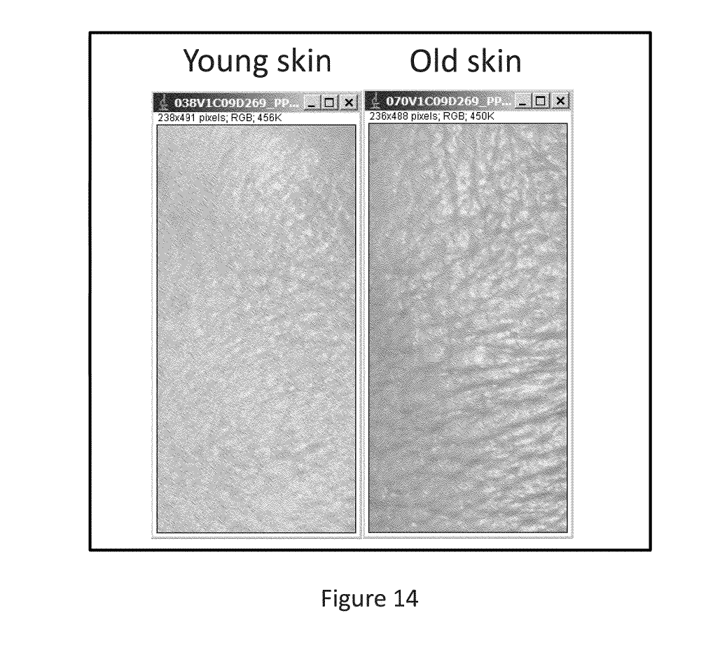

The skin surface texture parameter was measured from the parallel polarized images (e.g., FIG. 14, young skin in the left panel; old skin in the right panel).

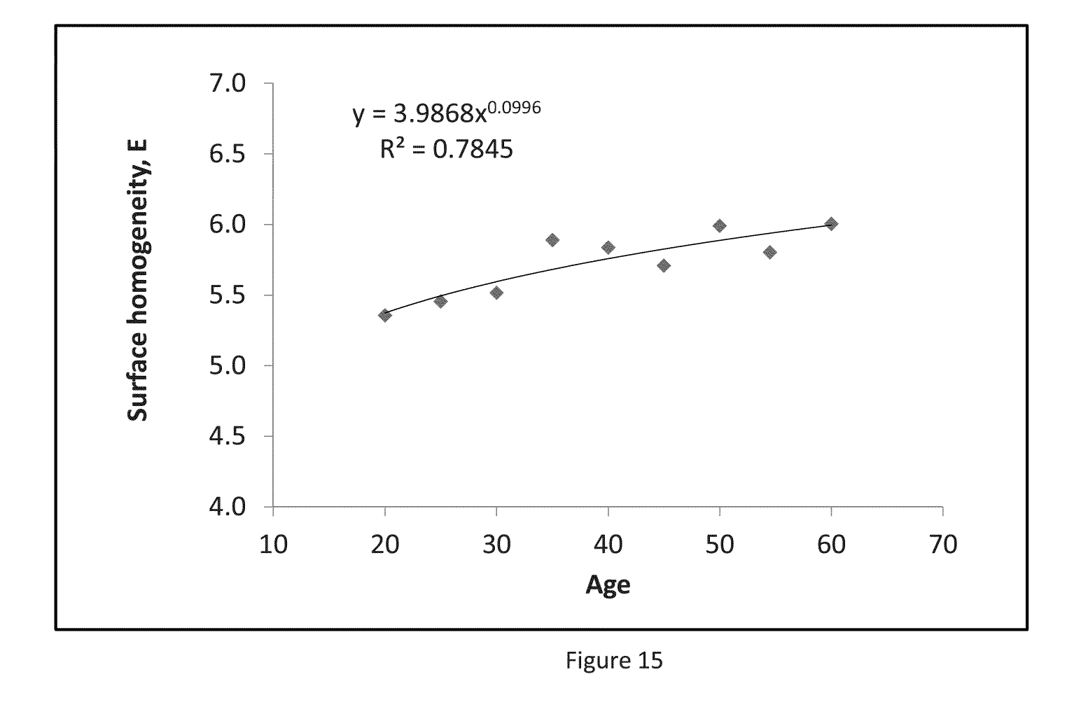

Skin surface homogeneity as described by Entropy is shown in FIG. 15.

5. Statistics:

Statistically significant correlation with age was observed for each of the nine measured visual properties.

6. Confirming the Age Dependency of 9 Objectively Measured Visual Skin Parameters:

Each measured skin parameter was examined for linearity with chronological age. Any parameter with a Pearson correlation coefficient greater than the critical value was accepted for use with the mathematical model of the present invention.

Statistically significant correlation was observed for each of the nine measured visual skin parameters, as discussed above.

The total number of wrinkles increased exponentially with increasing age. This suggest that the total number of wrinkles reflects the number of wrinkles as well as their severity. In this concept a deep wrinkle would be represented by multiple wrinkles lying on top of each other in one wrinkle location.

The lightness of skin tone, as defined by ITA° decreased with increasing age in a roughly linear fashion.

Its component, b*, increased with age indicating that older people have more yellowing shin tone.

Also, younger people exhibited higher translucency as the colors from the subsurface of skin were more visible due to less specular reflection.

Regarding the change in facial skin color unevenness with age, older people have more uneven skin tone.

Also, the number of visible facial pores increased with age.

It was found that the increase in the wrinkle score had the most prominent effect over age followed by the subsurface spots, pores, and skin yellowness.

8. Creating Standard Reference Curve:

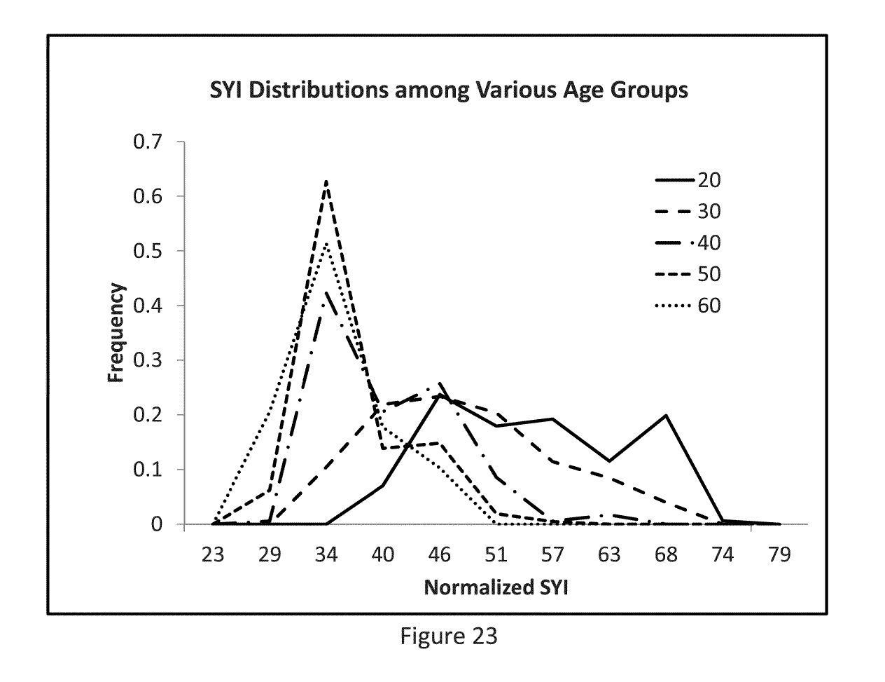

Following the image analysis, an SYI value was determined for each of the studied subjects. The distribution of SYI values for five age groups is shown in FIG. 23. As illustrated in FIG. 23, the younger age groups showed a wider distribution than the older age groups. Based on these values, a standard reference curve was generated and is shown in FIG. 24.

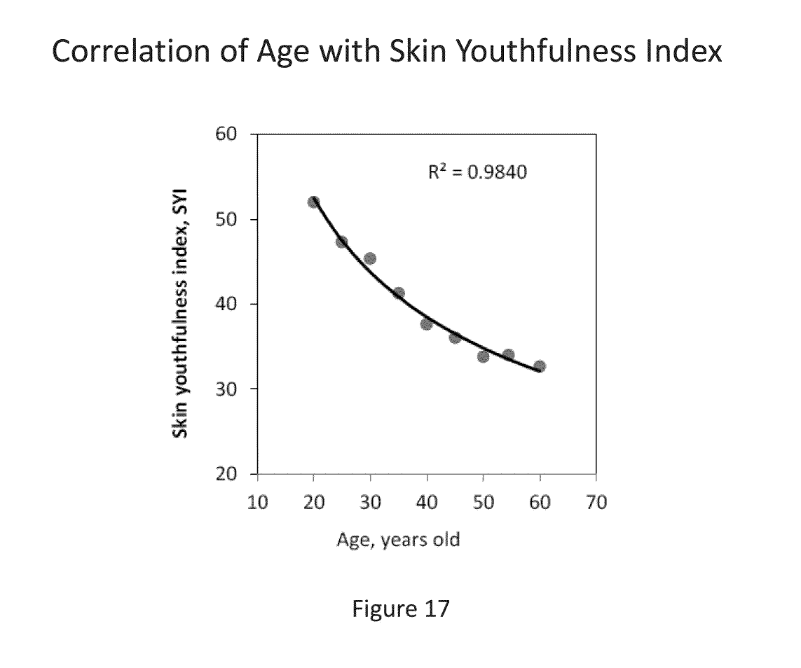

The SYI was then calculated using the nine objectively measured visual skin parameters. The factor is characterized in that the better the linearity of a measured parameter, the greater is its contribution to the SYI. It resulted in a downward power function between age and SYI, with a significance value of r²=0.984, as shown by the age-SYI plot in FIG. 17.

When the SYI values were converted from the above correlation back to estimated ages and then compared with the chronological ages of the test population, the observed differences between the chronological age and its corresponding estimated or apparent age was quite small. The mean error sum of squares (ESS), a statistical measure of goodness-of-fit, was 3.6 as opposed to 23.7 when the conventional multivariate linear regression method was used, which indicates a more powerful correlation method for this invention.

As seen in FIG. 17, the decrease in SYI was more dramatic among younger age groups and it leveled off after individuals reached 50 years of age.

The differences in the SYI values between two adjacent age groups were statistically significant for people up to 45 years old, as indicated by the p values of t-test in Table I.

TABLE I
T-test: p values of SYI comparison between various age groups
(N = 1505)

| Age | 20 | 25 | 30 | 35 | 40 | 45 | 50 | 55 | 60 |
|---|---|---|---|---|---|---|---|---|---|
| 20 | NA | | | | | | | | |
| 25 | 0.000 | NA | | | | | | | |
| 30 | 0.000 | 0.000 | NA | | | | | | |
| 35 | 0.000 | 0.000 | 0.000 | NA | | | | | |
| 40 | 0.000 | 0.000 | 0.000 | 0.000 | NA | | | | |
| 45 | 0.000 | 0.000 | 0.000 | 0.000 | 0.035 | NA | | | |
| 50 | 0.000 | 0.000 | 0.000 | 0.000 | 0.000 | 0.005 | NA | | |
| 55 | 0.000 | 0.000 | 0.000 | 0.000 | 0.000 | 0.004 | 0.776 | NA | |
| 60 | 0.000 | 0.000 | 0.000 | 0.000 | 0.000 | 0.000 | 0.119 | 0.033 | NA |

Count of volunteers in each age group:

| | | | | | | | | |
|---|---|---|---|---|---|---|---|---|
| 156 | 275 | 201 | 194 | 175 | 135 | 100 | 162 | 107 |

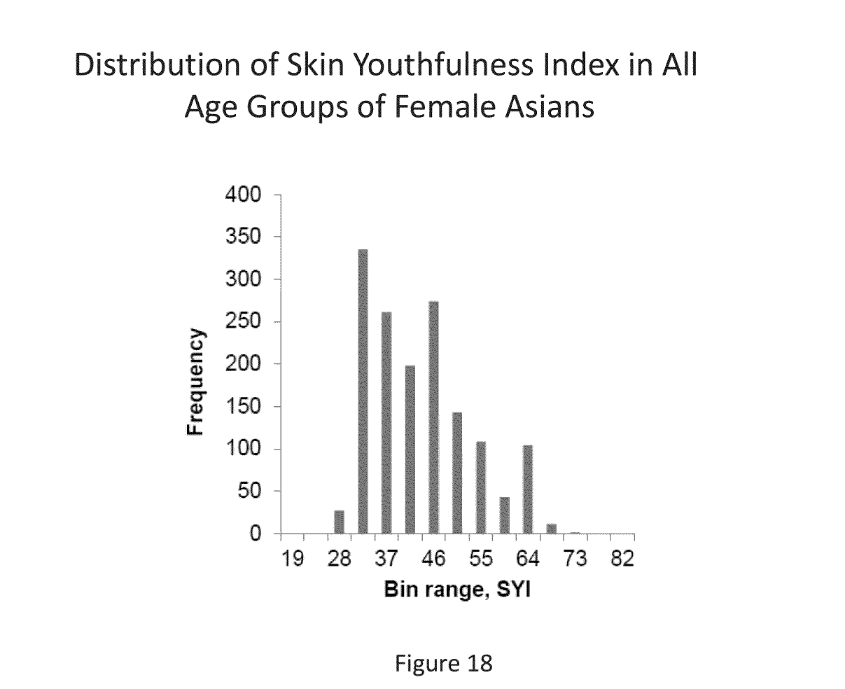

The distribution of the test population (1505 subjects) SYI is shown in FIG. 18. It ranged approximately from 25 to 75 with higher values indicating a more youthful skin.

Furthermore, when the SYI distribution was plotted (data not shown), it was observed that, due to individual differences in skin conditions such as skin color, wrinkles, and pores, the SYI values had a wide span in each age group but, on the other hand, showed relatively less variation in their SYI values.

The method and correlation described in this study have a great potential for product efficacy evaluation. For a given clinical study, the method allows to analyze the before and after clinical images of patients undergoing a cosmetic or medical treatment to objectively measure the nine visual skin parameters. If a treatment, product or a skin care regimen were to show a skin benefit such as, e.g., a wrinkle reduction or an increase in skin translucency, the change would be detected by the image analysis and show a positive change in its corresponding measurement results. When all benefits are combined in Equation 1, an increase in SYI would be obtained. Using FIG. 17, this increase in SYI value could be translated into a corresponding younger age group. Since all measured parameters are visual properties of facial skin, this measured increase in SYI suggests improvement in skin characteristics and more youthful-looking skin.

In conclusion, the large amount of images obtained from general public around the world allowed for objective measuring of different visual properties of facial skin in nine chronological age groups. Statistically significant correlation was obtained between chronological age and each visual parameter. Combining the different visual parameters into a single parameter enabled creation of an index of skin youthfulness for quantitative description of facial skin in any subject. An excellent correlation was obtained between chronological age and the SYI.

### Example 1

Using image analysis of facial images, 9 visual parameters: wrinkles, number of deep layer dark spots, visible pores, skin translucency, skin yellowness, skin redness, skin tone lightness, ITA°, evenness of skin tone, and the skin surface texture parameter (entropy, a measure of homogeneity of surface texture property) were measured in 1505 volunteers. Through statistical analysis, each of the measured skin parameters correlated with age with statistical significance.

Weight Factors for each of the measured visual parameters were calculated and results are shown in Table II below.

TABLE II
Calculation of Weight Factors:

| | | | | Sig | | +/− | Impact | Wt |
|---|---|---|---|---|---|---|---|---|
| Parameter | R² | r | R_{c, 2.05(n = 9)} | Co | % MaxImp | Correlation | Factor | Factor |
| Wrinkles | 0.9756 | 0.988 | 0.666 | 0.952 | 184% | −1 | 1.752 | 0.369 |
| Spots | 0.8661 | 0.931 | 0.666 | 0.750 | 133% | −1 | 1.001 | 0.211 |
| Pores | 0.9453 | 0.972 | 0.666 | 0.894 | 66% | −1 | 0.594 | 0.125 |
| STI | 0.9440 | 0.972 | 0.666 | 0.891 | 29% | 1 | 0.256 | 0.054 |
| a* | 0.8485 | 0.921 | 0.666 | 0.720 | 35% | −1 | 0.250 | 0.053 |
| b* | 0.9190 | 0.959 | 0.666 | 0.844 | 43% | −1 | 0.361 | 0.076 |
| ITA | 0.9338 | 0.966 | 0.666 | 0.872 | 24% | 1 | 0.211 | 0.044 |
| CUE | 0.9690 | 0.984 | 0.666 | 0.939 | 26% | −1 | 0.241 | 0.051 |
| Entropy | 0.7854 | 0.886 | 0.666 | 0.617 | 13% | −1 | 0.081 | 0.017 |
| IDM | 0.0000 | 0.000 | 0.666 | 0.000 | 38% | 1 | 0.000 | 0.000 |
| Contrast | 0.0000 | 0.000 | 0.666 | 0.000 | 19% | 1 | 0.000 | 0.000 |
| Total | | | | | | | 4.746 | 1.000 |

A standard reference curve between age and SYI was constructed (subjects for this study were selected at exact or almost exact chronological ages).

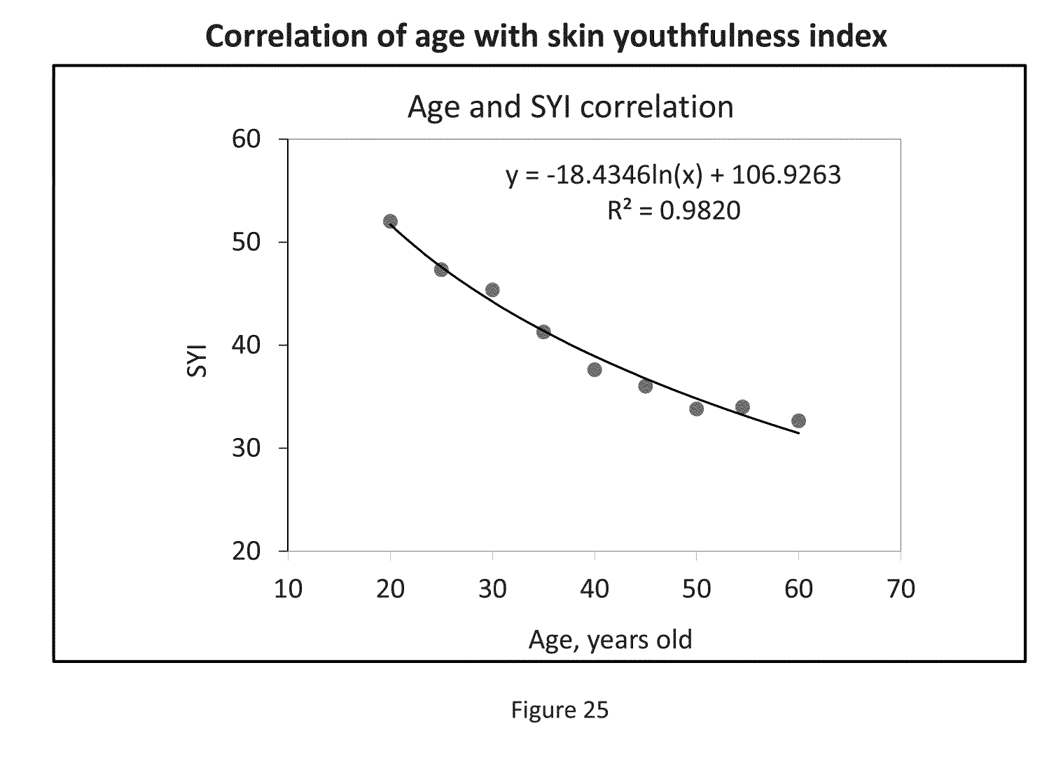

Excellent correlation with the r² value of 0.982 was obtained, as shown in FIG. 25.

The model was then used to predict apparent age using measured visual parameters. A good prediction was achieved with an error term (sum of error squares) of 3.6 for the data set.

### Example 2

Two data sets were used to validate the model of the present invention. The first set used VISIA-CR images of 104 Asian females whose age was exactly 28 years. Their average SYI value was 49.5, which corresponds to an estimated age of 23, a 5 year difference as compared to the actual chronological age. The second set used VISIA-CR images of 70 Asian females whose age was exactly 38 years. Their average SYI value was 41.8, which corresponds to an estimated age of 33.3, a 4.7 year difference as compared to the chronological age of the studied women.

### Example 3

One common anti-aging practice available to patients is the use of laser ablation to remove wrinkles and improve facial skin conditions. Such procedure is typically performed by dermatologists in their offices or clinics. While the patients accept the outcome, typically, an objective method of evaluating the effects of the treatment is not available to the patients.

Laser ablations were performed to reduce wrinkles from the faces of the volunteers.

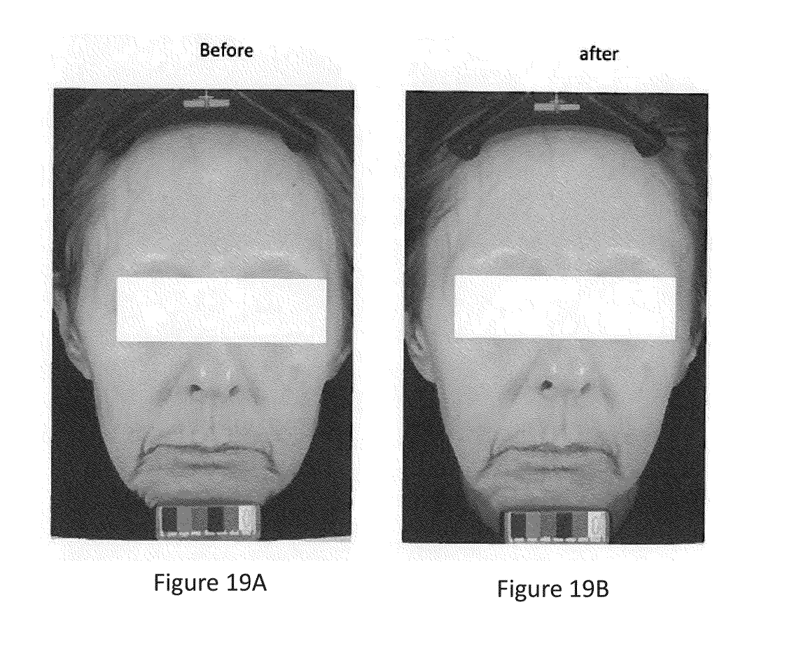

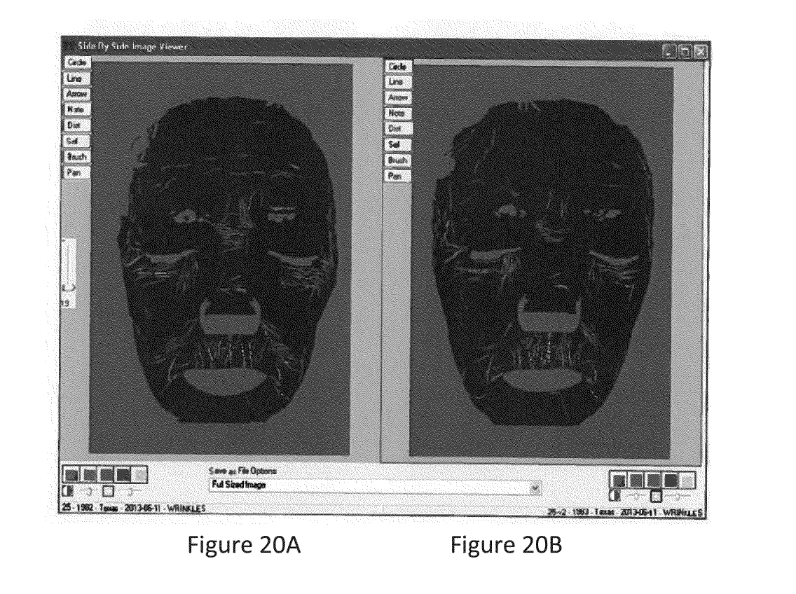

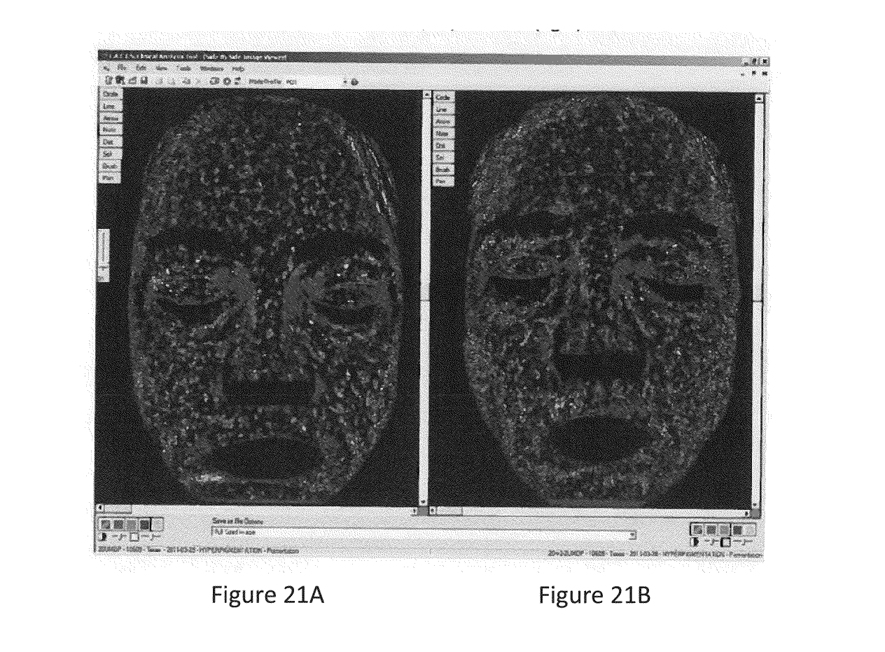

FACES images of the volunteers were taken before and after the laser ablation treatments. FIGS. 19A-19B show facial images of a patient before (FIG. 19A) and after (FIG. 19B) laser ablation treatment. The images were analyzed for 9 visual skin parameters. For example, as shown in FIGS. 19A-B, facial wrinkles were analyzed and measured before (A) and after (B) the laser ablation treatment; as shown in FIGS. 20A-20B, skin deep layer spots before (FIG. 20A) and after (FIG. 20B) the laser ablation treatment were measured and analyzed; as shown in FIGS. 21A-21B, the skin deep layer spots before (FIG. 21A) and after (FIG. 21B) treatment were analyzed and measured. The remaining 6 parameters were also measured and analyzed (not shown). The data obtained based on the measured 9 visual skin parameters were then incorporated into the SYI algorithm (equation 1) to determine the before and after SYI value for the patient.

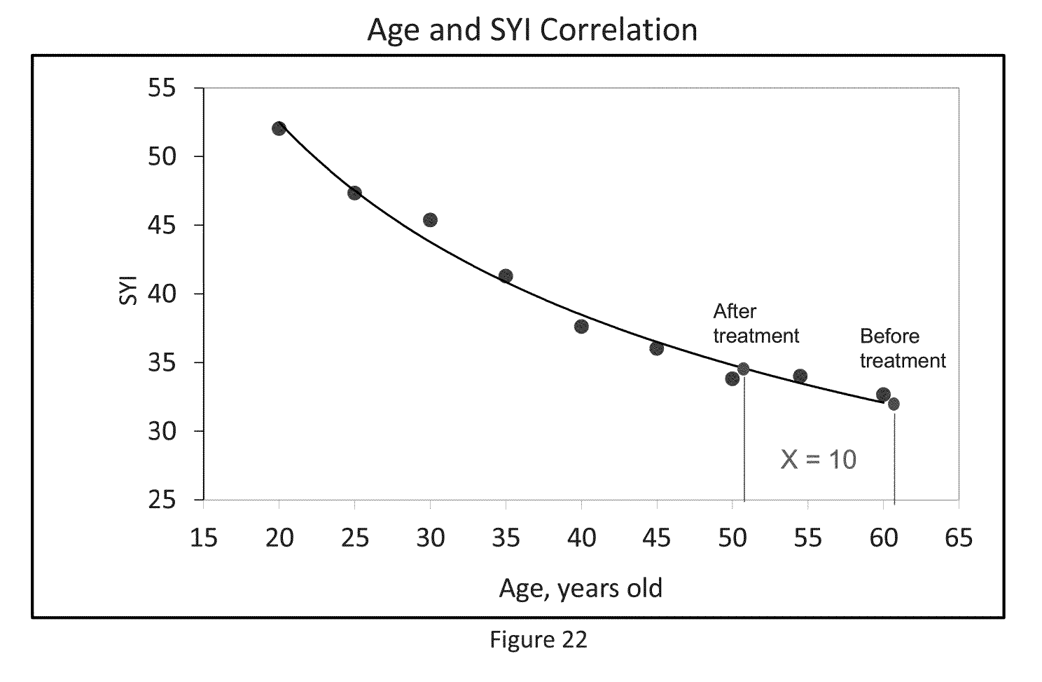

It was found that the SYI increased from 35.3 measured before the treatment (baseline measurement) to 37.5 measured after the laser ablation treatment to achieve a change in SYI of 2.2. This change equates to achieving skin characteristics 10.1 years younger according to age correlation of the mathematical model (FIG. 22).

### Example 4

To determine whether there was any improvement in facial skin characteristics/appearance following an Amelan, which is a powerful dermatological procedure that treats melasma symptoms, FACES images were obtained and analyzed for a patient undergoing Amelan procedure. Images of the patient were obtained and analyzed from before and after the procedure. Specifically, 9 visual facial skin parameters were analyzed for the patient.

The data obtained based on the measured 9 visual facial skin parameters were then incorporated into the SYI algorithm (Equation 1) to determine the before and after SYI values for the patient.

It was found that the SYI increased from 35.0 measured before the treatment (baseline measurement) to 39.4 measured after the Amelan procedure to achieve a beneficial change in SYI. This change equates to achieving skin characteristics 18.7 years younger according to age correlation of the mathematical model.

In additional embodiment, a skin youthfulness index (SYI) is used to establish a mathematical model that correlates age with the visual properties of facial skin using image analysis method. Images of 1,505 Asian female volunteers between the ages of 20 and 60 years were captured using VISIA-CR® system under five different lighting conditions. Skin properties, such as wrinkles, hyperpigmentation, pores, color, translucency, ITA°, color evenness, and surface texture parameters, were objectively measured from the images using image analysis algorithms.

Correlations between the measured parameters and the participants' chronological age were observed with statistical significance. By defining and calculating a set of weight factors, five objectively measured visual parameters of skin were found to be most relevant to describe skin conditions influenced by the aging process. Combining these parameters in a mathematical model we have established a skin youthfulness index which has a range of 0 to 100 and is inversely correlated to people's chronological age (R²=0.9959). The index allows us to accurately assess a person's apparent skin age based on the measured skin parameters. Of the various age groups tested, the largest difference between the actual and the calculated skin age was 2.4 years with a mean difference of 0.86 year. This model has potential for quantification of skin care product efficacy and thereby substantiation of new product claims.

### Example 5

### Facial Imaging System

A VISIA®-CR System (Canfield, U.S.A.) was used to capture facial images under five different lighting conditions (standard, flat, UV, cross polarized, and parallel polarized). The system consists of a facial imaging booth with eight flashes placed at different locations for uniform illumination, a Nikon 200 SLE camera, and a set of standard color plates. The camera settings were ISO 100, f14, and “cloudy” for white balance. Software was used to control the image capture process.

Study Design

From an image database of more than 30,000 participants that was collected around the world, the images of 1,505 female volunteers between the ages of 20 and 60 years, covering four Asian countries in the East, Far-East, and South-East regions were used. The selection was made to have exact or almost exact ages in each of nine age groups (20, 25, 30, 35, 40, 45, 50, 55, and 60 years old). An average of 167 subjects in each age group were included to ensure adequate representation of skin property distribution. Table III summarizes the age and the count of the volunteer population included in this study.

TABLE III
Age and count distribution of the study participant population

| Age | 20 | 25 | 30 | 35 | 40 | 45 | 50 | 54.5 | | 60 | | |
|---|---|---|---|---|---|---|---|---|---|---|---|---|
| Specific | 20 | 25 | 30 | 35 | 40 | 45 | 50 | 54 | 55 | 59 | 60 | 61 |
| Age | | | | | | | | | | | | |
| Count | 156 | 275 | 201 | 194 | 175 | 135 | 100 | 78 | 84 | 26 | 51 | 30 |

| | | | | | | | | | |
|---|---|---|---|---|---|---|---|---|---|
| Total | 156 | 275 | 201 | 194 | 175 | 135 | 100 | 162 | 107 |
| Count | | | | | | | | | |

All participants were confirmed by means of written informed consent. Five front view images of each study volunteer were taken during the image collection stage after face washing by a standardized cleansing procedure. Using proprietary image analysis software, visual skin properties representative of aging (wrinkles, pores, translucency, redness, yellowness, ITA°, unevenness of skin tone, and surface texture parameters) were quantified from the set of captured images. Statistical analysis was performed using JMP® 10.0.0 statistical software (SAS Institute Inc.) with the multiple regression function.

Image Analysis

Parameters Analysis

Image analysis algorithms were used to objectively quantify facial skin properties such as wrinkle score, hyperpigmentation score, pore count, skin color parameters, lightness of skin tone, evenness of skin tone, skin translucency, and surface texture properties. All images were first color corrected using standard color plates embedded in each picture to achieve accurate measurements of skin color and other visual skin properties. The automatic feature recognition algorithms generate a facial mask that excludes eyebrows, eyes, nostrils, mouth, and terminal hair, rendering only the skin surface for accurate wrinkle, pore, and subsurface hyperpigmentation analysis. A representative graphic output of the facial masks is shown in FIGS. 1A-1C.

Facial wrinkle analysis was performed in the entire facial area. A wrinkle score was reported which reflected both the number of wrinkles and wrinkle severity; therefore, a deep wrinkle would be equivalent to multiple smaller wrinkles lying on top of each other in one location. Skin sub-layer hyperpigmentation was measured from the UV images in which areas with large amounts of melanin deposition were quantified to produce a hyperpigmentation score. Facial pores were quantified in the selected regions of interest (ROI) that include nose, upper lip, chin, the cheek areas close to the nose, and the portion of the forehead close to eyebrows. The output of the facial pore analysis included pore count and pore area.

Facial skin color was measured using the cross-polarized images. ROIs on the cheeks and forehead were created following automatic detection of the facial features such as hairline, eyes, eyebrows, nose, and mouth. Color parameters in the RGB color space were obtained from the ROIs and converted to the L*, a* and b* of CIELAB color space using in-house developed algorithms in Image J (National Institutes of Health). The skin individual typology angle (ITA°) was calculated using the measured L* and b* values. Unevenness of skin tone (U) was measured as the variance of pixel intensity in each ROI. Skin surface texture parameters were obtained from the ROI using the statistical method of Gray Level Co-occurrence Matrix (GLCM), a built in function of Image J. Two GLCM parameters, entropy (E) and inverse difference moment (IDM), were found to be the most relevant to describing the age related changes of skin texture properties. Entropy is a measure of the orderliness of the surface texture pattern. The skin with more fine lines and wrinkles often shows more regular parallel pattern and would therefore result in higher values of entropy. IDM, on the other hand, indicates the homogeneity of surface texture pattern. Uniform surface texture pattern such as that of a young skin would have a high value of IDM.

Skin translucency, a quality of facial skin greatly appreciated in Asian culture, was measured by using both cross polarized and parallel polarized images. Skin with high translucency is perceived by consumers to have flawless surface appearance, delicate texture, subtle subsurface reflection, and a rosy glow. Matsubara et al. described an image analysis method to quantify facial skin translucency [Matsubara, A., Differences in the Surface and Subsurface Reflection Characteristics of Facial Skin by Age Group, Skin Res. Technol., 18 (2012) 29-35]. A modified version of this method was employed by quantifying skin translucency through diffuse reflection, as opposed to specular reflection used in Matsubara's study, and defined a skin translucency index based on the average intensity value and its distribution in each of the RGB channels.

Data Analysis

Data Type and Range

Properties of the 10 objectively measured visual parameters of facial skin are summarized in Table IV. Those extensive properties such as wrinkles, sub-layer spots, and pores were measured from the whole face area, while those intensive properties such as color and texture were measured from regions of interest on both cheeks.

TABLE IV
Properties of the objectively measured
visual parameters of facial skin

| | | | Mean value |
|---|---|---|---|
| Parameter | Name | Data range | (20-60 years old) |
| Wr | Wrinkles | 0-600 | 80 |
| S | Sub-layer spots | 10-100 | 166 |
| P | Pores | 10-4000 | 1632 |
| T | Skin translucency | 10-300 | 39 |
| a* | Redness | 9-30 | 16 |
| b* | Yellowness | 13-34 | 24 |
| ITA° | Skin tone lightness | 6-63 | 42 |
| U | Color unevenness | 10-200 | 65 |
| E | Texture orderliness | 3-7 | 6 |
| IDM | Local homogeneity | 0.3-0.7 | 0.5 |

Multiple Regression Analysis

A multiple regression analysis was performed using JMP® to correlate participant age with the objectively measured skin parameters in order to establish a linear equation in the following form:

$$Predicted Age = I + {\sum }_{i = 1}^{n} {C}_{i} {V}_{i} \qquad Equation 9$$

where I=intercept; C=coefficient; V=value of an objectively measured visual parameter; i=any specific parameter.

Skin Youthfulness Index

In addition to the multiple regression method, we established a new model, a skin youthfulness index (SYI), by correlating the age of the study participants with those parameters of their facial skin. The following were considered to define the SYI:

a single comprehensive index that indicates the youthfulness of facial skin and is correlated inversely with people's chronological age (i.e., younger people have a higher index value and older people a lower value);

an index that is affected by the measured visual parameters in a linear composite fashion through appropriately defined weight factors;

the positive or negative effect of each parameter on the index is reflected (i.e., the value of a parameter that increases with age would have a negative influence on SYI, whereas the value of a parameter that decreases with increasing aging would have a positive effect);

the index would have a target scale of 0-100.

Using these considerations, the following linear composite function was proposed:

$$SYI = {N}_{1} ( {N}_{2} + {\sum }_{i = 1}^{n} {JW}_{i} ( ln {V}_{i} ) ) \qquad Equation 10$$

where W=weight factor; V=value of an objectively measured visual parameter; i=any specific parameter type, and the constants N₁ and N₂ were factors to produce SYI values on a scale of 0-100. The J term in Equation 10 indicates whether a parameter has a positive or negative effect on SYI; J=1 for a positive effect and J=−1 for a negative effect. For example, since a higher age indicates a lower SYI value, an increasing wrinkle score with increasing age would have a negative effect on SYI.

Weight Factor Calculation

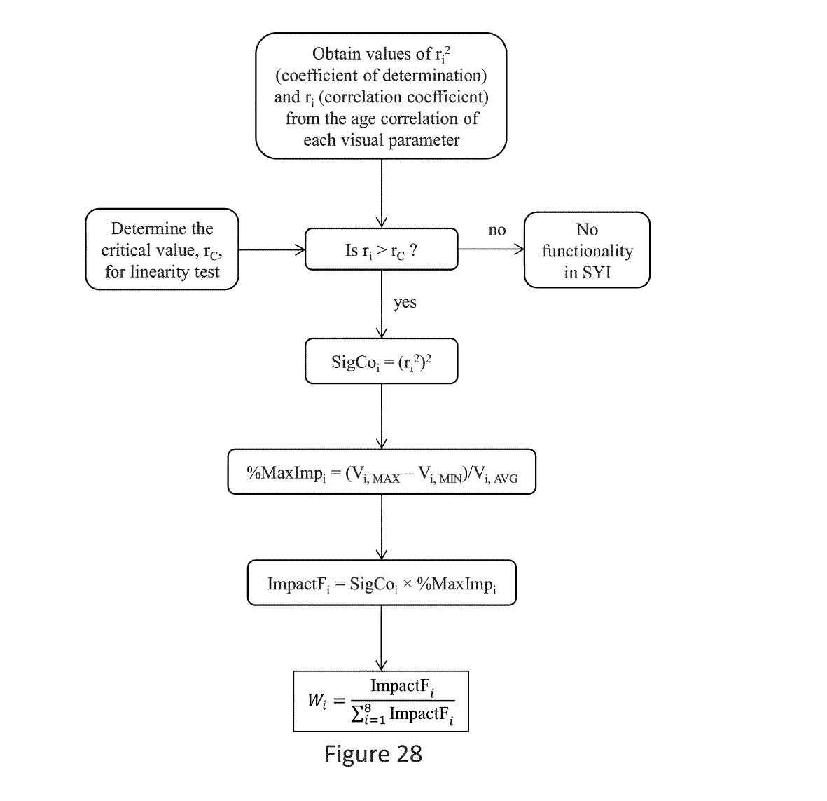

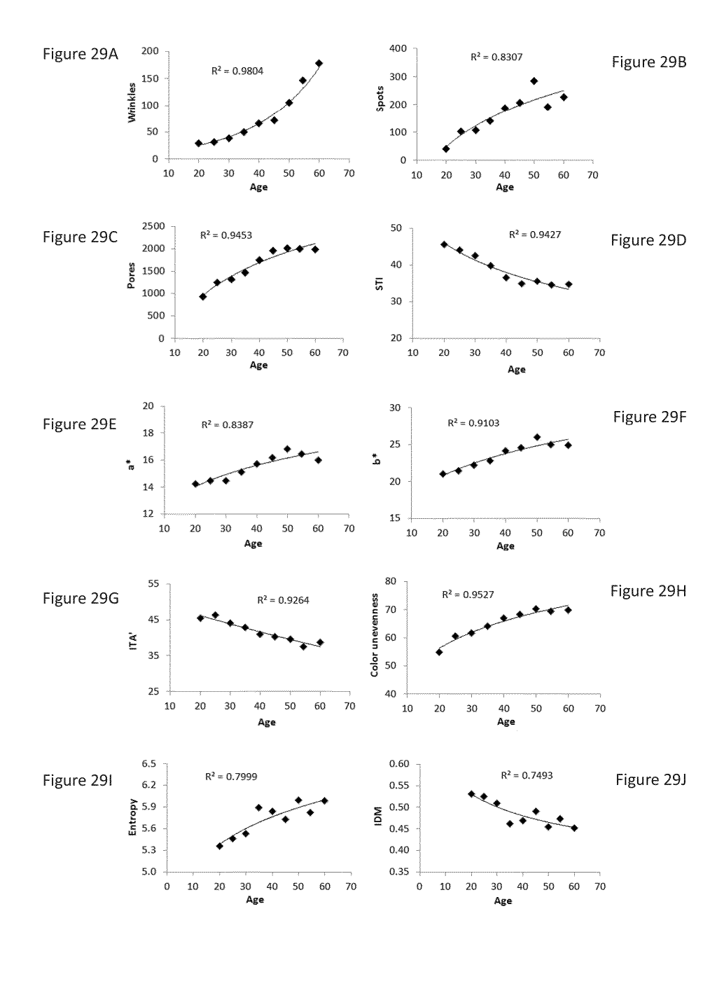

Calculating the weight factor W for each visual parameter was a key step in the development of the index and was performed as outlined in the flow chart shown in FIG. 28. Specifically, the coefficient of determination, rᵢ², and the correlation coefficient, r, were obtained from the age correlation plots for each of the 10 visual parameters (FIGS. 29A-29J). A linearity test was conducted by determining a critical value for the correlation coefficient [Weathington, B., Cunningham, C., and Pittenger, D. (Eds.), Understanding Business Research, John Wiley & Sons, Inc, Hoboken, N.J., 2012, pp. 245-270]. If the correlation of a parameter passed the linearity test, the variable was considered meaningful and was included for the weight factor calculation. A significance factor was then defined, SigCo=(r²)², which ranks the significance of contribution of the ten visual parameters. Then, a maximum impact factor (% MaxImp) was defined, emphasizing the level of influence a variable has as it changes with age (i.e., a high % MaxImp indicates that the parameter has a high impact on the SYI-age correlation). Then the impact factor, defined as ImpactF, was calculated as the product of SigCo and % MaxImp. The weight factor was finally calculated by normalizing the impact factor in a unit fraction form.

Age Prediction from SYI

After a function of SYI was obtained, it was then correlated with the study participants' chronological age to establish a SYI-age curve. Such a curve enables one to examine the goodness of fit of Equation 10 by computing the residual sum of squares between each group's actual and calculated age. In addition, this SYI-age correlation allows one to calculate a person's skin age from the objectively measured visual parameters of facial skin, as discussed later in the results and discussion.

Parameter Optimization

To identify parameters that contribute most meaningfully to SYI a statistical parameter, residual sum of squares (RSS), was used to determine the goodness of fit in the SYI-age correlation. Using Equation 10, the individual effect of each parameter was first evaluated to identify the one which correlated the best with age. The combined effects were then examined by adding other parameters one after another to Equation 10. Their corresponding RSS values were calculated and compared to determine if the age correlation was improved.

Results and Discussion

Effect of Age on the Measured Visual Parameters of Skin

It has been well documented through clinical grading that a person's visual signs of aging increase with age [R. Bazin, and F. Flament, Skin Aging Atlas Volume 2 Asian Type, Paris: Editions Med'Com, 2010, pp. 28].

In this study, a statistically significant age correlation for each of the ten objectively measured visual properties (wrinkles, pores, translucency, redness, yellowness, ITA°, unevenness of skin tone, and surface texture) was observed. FIGS. 29A-29J shows the average values of each of the visual parameters plotted against the participants' chronological age. The average wrinkle score increased exponentially with increased age (FIG. 29A). Since the algorithms include both number and severity of facial wrinkle in the calculation, a deep and wide wrinkle is represented by multiple single wrinkle lines as opposed to a single line color coded to differentiate it from other smaller wrinkles, as seen in many commercial wrinkle-analysis software packages. It is believed that an exponential increase in facial wrinkling over age displays a meaningful progression of aging process of human facial skin.

FIG. 29B indicates that the amount of sub-layer spots increases with age. This is due to the accumulative UV damage acquired during life.

FIG. 29C shows the average number of visible pores, which increases steadily with age and plateaus after age 45. While it is difficult to argue that pore number increases with age in a physiological sense, we concluded that, due to changes in skin color and pore size, facial pores become more easily detectable with age, both visually and with image analysis.

Facial skin translucency decreases with increasing age (FIG. 29D) and levels off after age 45. Younger people possess higher skin translucency as their skin looks less dull and exhibits higher diffuse reflection. Therefore, the color components in the subsurface of skin are more visible in younger people.

The lightness of skin tone, as defined by ITA°, decreases steadily with increasing age (FIG. 29G), indicating that older people have darker complexions, which agrees with the trend of changing facial skin color in a Caucasian population [Zedayko, T., Azriel, M., and Kollias, N., Caucasian Facial L* Shifts May Communicate Anti-Ageing Efficacy, Int. J. Cosmet. Sci., 33 (2011) 450-454]. One component of ITA°, b* which is a measure of skin yellowness, increases with age (FIG. 29F), indicating that older people in general have more yellowish skin tone.

A similar trend is observed for skin redness as shown by the a* values in FIG. 29E.

Age also increases the unevenness of facial skin tone which becomes less even with age due to discoloration, wrinkling, and other physiological changes (FIG. 29H).

The local homogeneity of skin texture (IDM) decreases with age while the orderliness of skin texture (entropy) exhibited the opposite trend, as shown in FIGS. 29J and 29I.

Age Correlation by Multiple Regression Analysis

The values of ten objectively measured visual parameters were fitted to Equation 9 using the multiple regression tool in JMP®. After examining the outcome of the analysis, three parameters (STI, b*, and IDM) which had p-values larger than 0.05 were removed from the correlation. The final linear equation obtained from the multiple regression analysis correlated the participants' age with seven parameters, with a r²=0.6277. The output of the multiple regression is shown in Table V. Inserting those values into Equation 9, the predicted age of the nine groups of Asian female volunteers using the average values of those visual parameters was calculated.

TABLE V
Estimated parameters (I & Cᵢ in Equation
I) using multiple regression analysis

| Parameters | Estimate, I & Cᵢ | Prob > \|t\| |
|---|---|---|
| I | 25.25077 | <.0001 |
| Wrinkles | 0.090014 | <.0001 |
| Spots | 0.008194 | <.0001 |
| Pores | 0.005913 | <.0001 |
| a* | −0.32862 | <.0001 |
| ITA′ | −0.3403 | <.0001 |
| CUE | 0.022603 | 0.0065 |
| Entropy | 2.33272 | <.0001 |

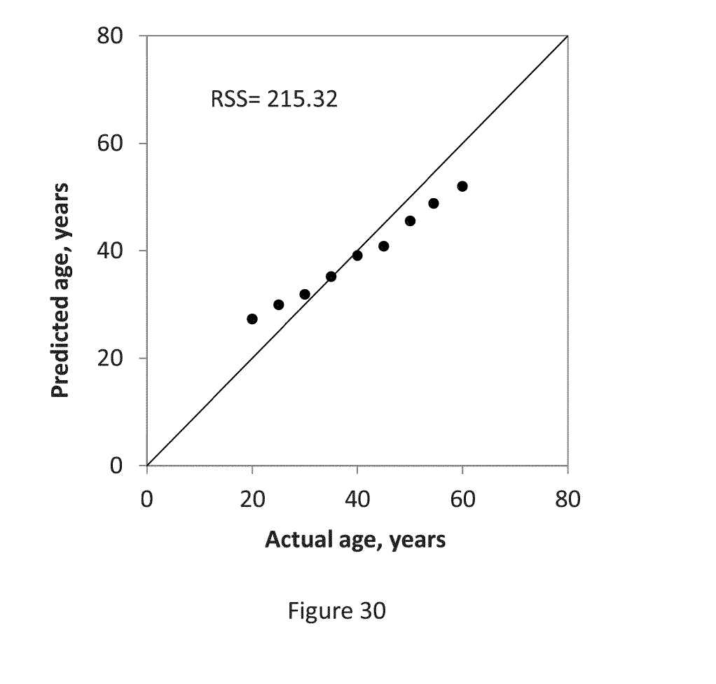

FIG. 30 shows the correlation between the actual age and the predicted age of each group with a RSS=215.32. Compared to the best fit line (diagonal), the predicted ages deviated more in the lower and higher age groups. The largest difference between the predicted and the actual age was 8.0 years.

Skin Youthfulness Index and its Correlation with Age

To calculate skin youthfulness index (SYI), the weight factors for each of those ten visual parameters by following the flow diagram described in FIG. 28 was calculated first. The results are shown in Table VI, from which we can see that skin wrinkling has the most significant effect on the SYI.

TABLE VI
Weight factors for Equation 10

| | | After |
|---|---|---|
| | Weight | Parameter |
| Parameter | Factor (W) | Optimization |
| Wrinkles, Wr | 0.435 | 0.627 |
| Sub-layer spots, S | 0.202 | 0 |
| Pores, P | 0.121 | 0.174 |
| Translucency, T | 0.056 | 0.081 |
| Redness, a* | 0.026 | 0 |
| Yellowness, b* | 0.038 | 0.056 |
| Skin tone lightness, ITA° | 0.044 | 0 |
| color unevenness, U | 0.043 | 0.062 |
| Texture orderliness, E | 0.015 | 0 |
| local homogeneity, IDM | 0.019 | 0 |
| Total | 1.000 | 1.000 |

This is due to the fact that it is closely correlated with age and its change over age is the largest in the order of magnitude. This is consistent with the common understanding that facial wrinkling is a significant marker for skin aging. Plugging the objectively measured visual parameters from each of the 9 age groups into Equation 10, a set of SYI values for the corresponding age groups was calculated. Then, by correlating the SYI with the volunteers' actual age, a linear function was obtained which allowed to back-calculate their apparent skin age based on their objectively measured visual parameters of skin. Table VII summarizes those results together with the difference between the predicted and the actual age of the study volunteers. The goodness of fit was calculated from this table and a RSS=22.96 was obtained, which is much better than that of the multiple regression method.

TABLE VII
Results of SYI and age calculation using Equations 2 & 3

| Average age | | Average Age | |
|---|---|---|---|
| (actual) | SYI | (calculated) | Difference |
| 20 | 73.8 | 18.0 | 2.0 |
| 25 | 70.7 | 26.2 | 1.2 |
| 30 | 69.5 | 29.2 | 0.8 |
| 35 | 67.0 | 35.9 | 0.9 |
| 40 | 64.6 | 42.3 | 2.3 |
| 45 | 63.4 | 45.4 | 0.4 |
| 50 | 61.1 | 51.6 | 1.6 |
| 55 | 60.3 | 53.6 | 0.9 |
| 60 | 58.9 | 57.3 | 2.7 |

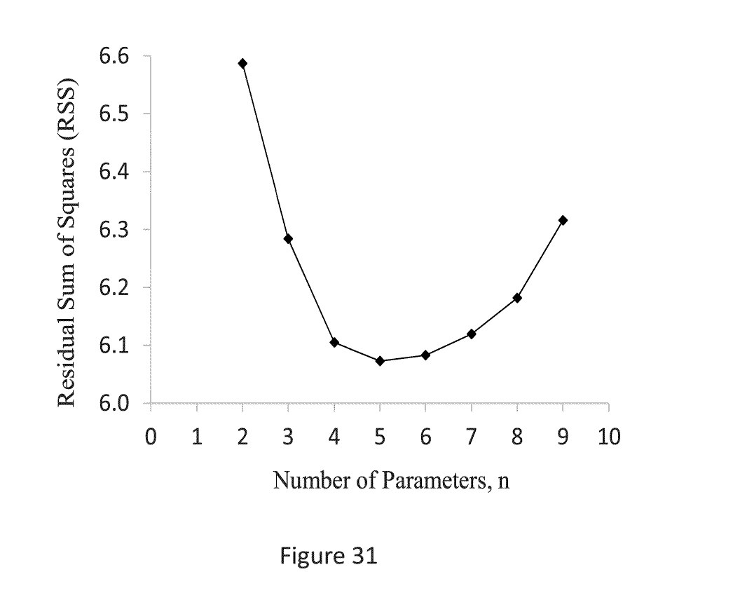

Using the parameter optimization method described above, the effect of each visual parameter and the combinations of various parameters which contribute to the SYI-age correlation was examined. This was done by finding the best age correlation (the least RSS) among the individual parameters and then adding more parameters one after another to identify the best combination at the next level. Among the ten individual visual parameters, the effect of wrinkle score correlated the best with age (RSS=12.61). Adding other parameters to wrinkle score and screening through all ten parameters at various combinations, we were able to obtain the optimal parameter combination as shown in FIG. 31. Based on the chart, by combining more parameters with wrinkles, better age prediction was achieved with decreasing RSS values until a point that adding more parameters started to influence the SYI-age correlation in a negative way. This optimal combination involved five parameters: wrinkle score, pores, skin translucency, yellowness, and color unevenness. Their corresponding weight factors are listed in Table IV above, under the column heading of “after parameter optimization”.

With the above results, we obtained the final equation for SYI calculation:

SYI=10(10+(0.081 ln T−0.627 ln Wr−0.174 ln P−0.056 ln b*−0.062 ln U) Equation 11

where T=translucency index, Wr=wrinkle score, P=pore score, b*=yellowness, and U=color unevenness.

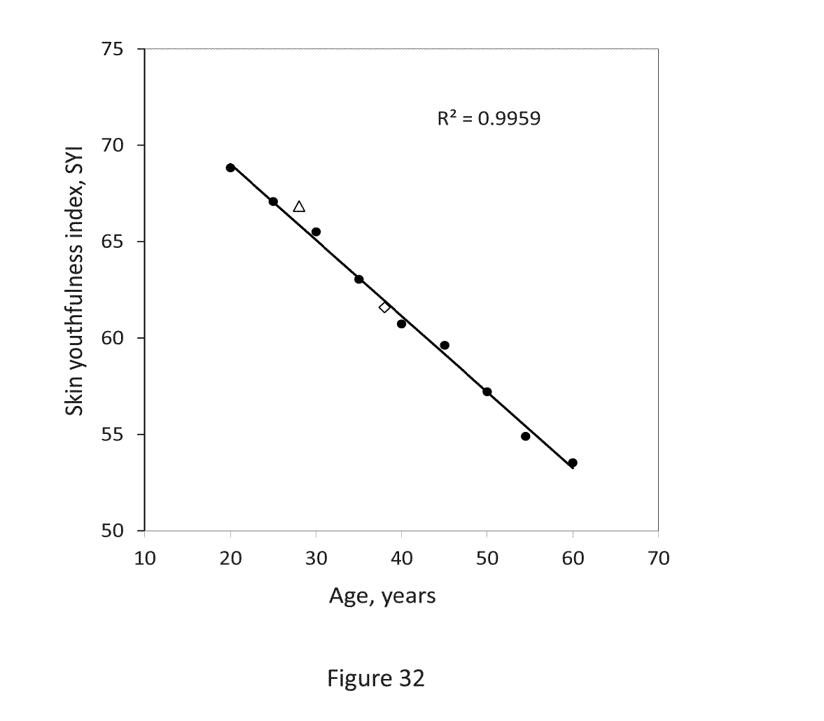

Using Equation 11 and the values of the objectively measured visual parameters of facial skin, the SYI values from the images of all 1,505 study participants in the nine age groups indicated above were calculated. The average SYI value of each age group was correlated with the chronological age of the study participants, as shown by the solid dots and the regression line in FIG. 32 from which a strong inverse linear correlation was observed with r²=0.9959.

As expected, the younger groups have higher SYI values while the opposite holds true for the older groups.

The correlation in FIG. 32 enabled to calculate a person's apparent age based on the visual parameters objectively measured from her facial images. By “apparent age,” it is meant the age of skin, which has visual properties of the facial skin of people in that specific age group. This age might be different from the perceived age as the latter is subjective in nature and is strongly influenced by a perceiver's knowledge, experience, preference, and culture background. Therefore, when Equation 11 is used to predict a person's age based on the measured visual parameters of facial skin, the subject exhibits a skin age similar to those people who typically possess the same level of visual properties. Higher levels of skin aging parameters shown in the facial images result in lower SYI values, which corresponds to a higher apparent age.

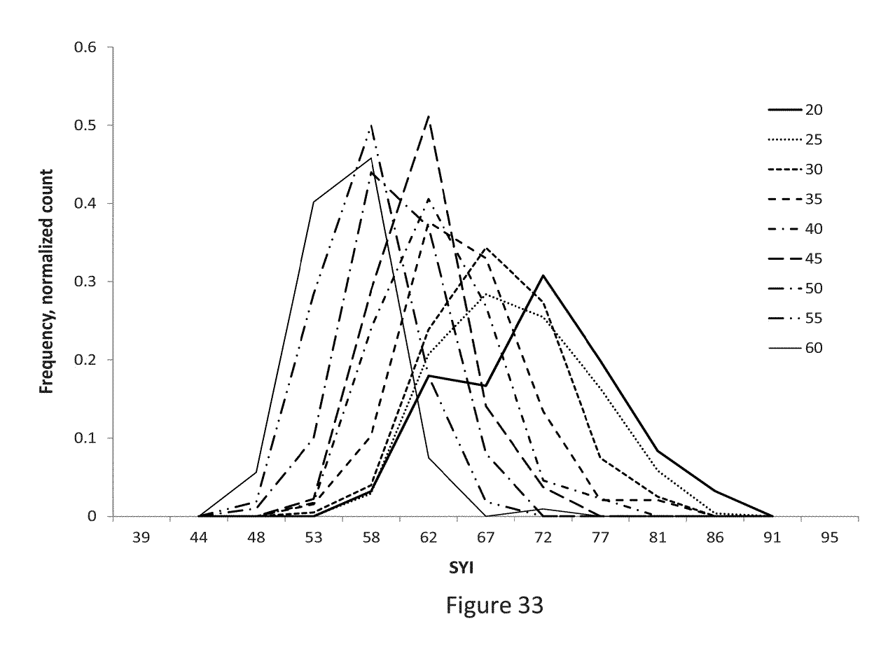

FIG. 33 shows the normalized SYI distributions for each of the nine age groups. The SYI values for all 1505 participants ranged approximately from 44 to 91, with higher values corresponding to a more youthful skin. A shown in FIG. 33, the SYIs for the 20 year old group reside in the high value region. With increase in group age, the SYI distributions shifted toward the lower value region diminishing the peak value from 72 down to 53. These distribution curves show how people's SYI, as well as their exhibited visual properties of facial skin, change with age.

Student's t-tests were performed to identify significant differences in SYI distributions between the different age groups. The differences in SYI values between any two adjacent age groups were statistically significant at a 95% confidence level, as shown by the p values in Table VIII. Since the study participants were selected who are at the exact age (or almost exact age) for each of the nine age groups indicated above, the results of this t-test become very meaningful. For example, from Table VIII, the skin's visual properties and its youthfulness index are statistically different between people of 20 and 25 years old. They are now measureable and distinctive properties of skin.

TABLE VIII
p values of SYI between various age groups

| Age | 20 | 25 | 30 | 35 | 40 | 45 | 50 | 55 | 60 |
|---|---|---|---|---|---|---|---|---|---|
| (N) | (156) | (275) | (201) | (194) | (175) | (135) | (100) | (162) | (107) |
| 20 | NA | | | | | | | | |
| 25 | 0.005 | NA | | | | | | | |
| 30 | <0.05 | 0.002 | NA | | | | | | |
| 35 | <0.05 | <0.05 | <0.05 | NA | | | | | |
| 40 | <0.05 | <0.05 | <0.05 | <0.05 | NA | | | | |
| 45 | <0.05 | <0.05 | <0.05 | <0.05 | 0.017 | NA | | | |
| 50 | <0.05 | <0.05 | <0.05 | <0.05 | <0.05 | <0.05 | NA | | |
| 55 | <0.05 | <0.05 | <0.05 | <0.05 | <0.05 | <0.05 | <0.05 | NA | |
| 60 | <0.05 | <0.05 | <0.05 | <0.05 | <0.05 | <0.05 | <0.05 | 0.001 | NA |

Validation

To validate the age predictability of Equation 11, two new data sets were selected from a Southeastern Asian population. Facial images of 104 female volunteers at 28 years of age and 70 females at 38 years of age were analyzed. The five visual parameters were measured and inserted into Equation 11 along with their corresponding weight factors shown in Table VI. The resulting SYIs are shown by the hollow square and triangle, respectively in FIG. 32. While both of the validating data points fit into the model well, the average value of SYI for the 38 year old group lies almost right on the regression line suggesting an excellent model for this analysis.

Age Prediction Using Measured Visual Parameters of Skin

From the results of these analyses, we were able to calculate skin age using the objectively measured visual parameters of facial skin. This was done by re-plotting the data in FIG. 32 to show a dependence of age on SYI. Fitting the correlation to a linear model we obtained the following for the prediction of a person's apparent age:

Age=194.38−2.26SYI Equation 12

where SYI=skin youthfulness index calculated from Equation 11, and Age=the apparent age of any study participant.

Inserting the average SYI values into the equation allowed to calculate the average age of the nine age groups.

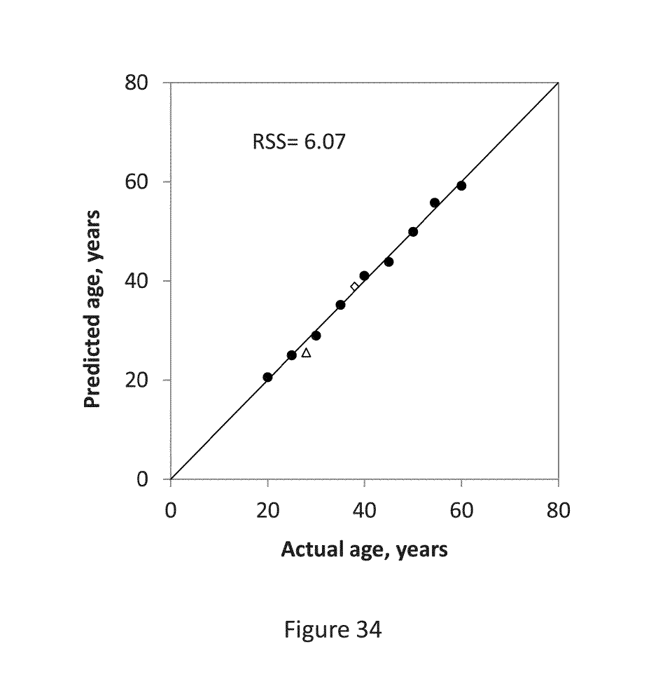

FIG. 34 is a correlation plot in which the predicted ages are plotted against the actual ages of the nine test groups. An excellent correlation was obtained with RSS=6.07. Comparing the result of this age correlation with that of the multivariate regression analysis (FIGS. 29A-J), the SYI method is much more effective at predicting the skin age of the population in this study than the conventional multiple regression method. The maximum age deviation between the predicted and the actual ages was 1.3 years, much smaller than the 8.0 year deviation resulted from the multiple regression method for the same population.

The results from the SYI analysis also show good age correlation and suggest that SYI can be used for meaningful age prediction. Using the data from the 28 and 38 year age groups used for model validation, the apparent ages were calculated to be 25.6 and 38.9, respectively. As indicated in FIG. 34, the differences of 2.4 and 0.9 years between the actual and the calculated ages for the 28 and 38 year age groups, respectively, suggest a fairly good age prediction capability.

Concept Application

The SYI-age correlation described in this study may provide a useful method for the evaluation of skin care product efficacy. For any given clinical study, one would be able to analyze both before and after clinical images to objectively measure the five visual parameters. If a product or skin care regimen were to demonstrate a skin benefit, such as wrinkle reduction or increase in skin translucency, it would be detected by image analysis and show a positive change in its corresponding measurement results.

When the improved values are inserted into Equation 11, the corresponding SYI value show an increase as seen in FIG. 32. This increase in SYI corresponds to a skin property of people of a younger age group, i.e., a decrease in calculated skin age. Since all measured parameters are the visual properties of facial skin, this decrease in calculated skin age after product treatment could be used to support a claim that the facial skin of an individual looked measurably years younger after the product use. The preliminary analysis on images before and after a laser resurfacing procedure had indicated very promising reduction in the calculated age after treatment (unpublished data).

### CONCLUSIONS

The large number of facial images obtained from Asian female consumers allowed the Applicants to objectively measure different visual properties of facial skin in nine age groups. Statistically significant age correlation was obtained for each of the measured visual parameters of skin. Combining the objectively measured parameters into a single function enabled us to establish a novel index of skin youthfulness (SYI), which quantitatively describes the aging conditions of facial skin. An excellent correlation was obtained between age and SYI providing a potentially useful application to establish skin product efficacy and to substantiate new product claims.

It is therefore intended that the foregoing detailed description be regarded as illustrative rather than limiting, and that it be understood that it is the following claims, including all equivalents, that are intended to define the spirit and scope of this invention.

1. A method of determining a Skin Youthfulness Index (SYI) of a subject, comprising:
(i) measuring visual skin parameters; and
(ii) calculating the SYI of the subject wherein the SYI has an inverse correlation to human age and describes a skin youthfulness of a human subject on a 0 to 100 scale with a higher value corresponding to a more youthful appearance than the chronological age of the subject.

2. The method of claim 1, wherein the step of measuring comprises measuring one to nine visual skin parameters.

3. A method of determining a Skin Youthfulness Index (SYI) of a subject, comprising:
(i) measuring visual skin parameters of the subject; and
(ii) calculating the SYI of the subject, wherein the SYI comprises:
SYI=10(10+(0.081 ln T−0.627 ln Wr−0.174 ln P−0.056 ln b*−0.062 ln U) Equation 11
wherein:
T corresponds to translucency index,
Wr corresponds to wrinkle score,
P corresponds to pore score,
b* corresponds to yellowness, and
U corresponds to color unevenness.

4. The SYI of claim 3, wherein the SYI has a target scale of 0-100, with a higher value corresponding to a more youthful appearance of the skin of a subject than the chronological age of the skin of the subject.

5. A method of claim 3, further comprising normalizing data from the measured visual skin parameters.

6. The method of claim 3, wherein the measuring step is conducted from an image.

7. The method of claim 3, further comprising photographing the subject under five different lighting conditions: standard, flat, UV or narrow-band blue light, cross polarized, and parallel polarized.

8. A method for measuring an improvement of skin characteristics following a treatment comprising:
(i) measuring visual skin parameters: wrinkle score (Wr), pore score (P), translucency index (T), skin yellowness (b*), and unevenness of skin tone (U) before (_{b}) and after (ₐ) the treatment;
(ii) determining a before-the treatment skin youthfulness index (SYI_{b}) value, wherein SYI_{b} is calculated:
SYI_{b}=10(10+(0.081 ln T_{b}−0.0627 ln Wrb_{b}−0.174 ln P_{b}−0.056 ln b*_{b}−0.062 ln U_{b}) (Equation 13)
(iii) determining an after-the treatment skin youthfulness index (SYIₐ) value, wherein the SYIₐ is calculated:
SYIₐ=10(10+(0.081 ln Tₐ−0.0627 ln Wrbₐ−0.174 ln Pₐ−0.056 ln b*ₐ−0.062 ln Uₐ) (Equation 14)
(iv) correlating the SYI_{b} value to an age standard reference curve to determine a before-the treatment age value;
(v) correlating the SYIₐ value to the age standard reference curve to determine an after-the treatment age value; and
(vi) determining the difference between the before-the treatment age value and the after-the treatment age value to measure improvement, if any, of facial skin characteristics following the treatment.

9. The method of claim 8, wherein improvement, if any, shows a positive change in its corresponding measurement results.

10. The method of claim 8, wherein the measuring step is conducted from an image.

11. The method of claim 8, further comprising photographing the subject under five different lighting conditions: standard, flat, UV or narrow-band blue light, cross polarized, and parallel polarized.

12. The method of claim 8, wherein the treatment comprises a cosmetic treatment.

13. The method of claim 8, wherein the treatment comprises a medical treatment.

14. A method of determining an apparent age of a subject using a smart device, comprising:
using a camera on a smart device to acquire an image of a subject;
sending the acquired image to a processor;
using a set of instructions that, upon execution by the processor, causes the processor to perform at least the following:
(i) using the acquired image to measure visual skin parameters: wrinkle score (Wr), pore score (P), translucency index (T), skin yellowness (b*), and unevenness of skin tone (U) before and after the treatment,
(ii) determining a before-the treatment skin youthfulness index (SYI_{b}) value, wherein SYI_{b} is calculated:
SYI_{b}=10(10+(0.081 ln T_{b}−0.0627 ln Wrb_{b}−0.174 ln P_{b}−0.056 ln b*_{b}−0.062 ln U_{b}) (Equation 13)
(iii) determining an after-the treatment skin youthfulness index (SYIₐ) value, wherein the SYIₐ is calculated:
SYIₐ=10(10+(0.081 ln Tₐ−0.0627 ln Wrbₐ−0.174 ln Pₐ−0.056 ln b*ₐ−0.062 ln Uₐ) (Equation 14)
(iv) correlating the SYI_{b} value to an age standard reference curve to determine a before-the treatment age value,
(v) correlating the SYIₐ value to the age standard reference curve to determine an after-the treatment age value, and
(vi) determining the difference between the before-the treatment age value and the after-the treatment age value to measure improvement, if any, of facial skin characteristics following the treatment; and
(vii) using a display to display a result of the determined difference.

15. A smart device comprising:
a display; and
a computer-readable medium having a set of instructions that, upon execution by a processor, causes the processor to perform at least the following:
(i) using one or more images of a subject to measure visual skin parameters: wrinkle score (Wr), pore score (P), translucency index (T), skin yellowness (b*), and unevenness of skin tone (U) before and after the treatment,
(ii) determining a before-the treatment skin youthfulness index (SYI_{b}) value, wherein SYI_{b} is calculated:
SYI_{b}=10(10+(0.081 ln T_{b}−0.0627 ln Wrb_{b}−0.174 ln P_{b}−0.056 ln b*_{b}−0.062 ln U_{b}) (Equation 13)
(iii) determining an after-the treatment skin youthfulness index (SYIₐ) value, wherein the SYIₐ is calculated:
SYIₐ=10(10+(0.081 ln Tₐ−0.0627 ln Wrbₐ−0.174 ln Pₐ−0.056 ln b*ₐ−0.062 ln Uₐ) (Equation 14)
(iv) correlating the SYI_{b} value to an age standard reference curve to determine a before-the treatment age value,
(v) correlating the SYIₐ value to the age standard reference curve to determine an after-the treatment age value,
(vi) determining the difference between the before-the treatment age value and the after-the treatment age value to measure improvement, if any, of facial skin characteristics following the treatment, and
(viii) causing the display to display a result of the determined difference.
